SEQUENCE LISTING (44 sequences)

SEQ ID NO: 1 - DNA, 21 bp, synthetic construct
  source 1..21: mol_type = other DNA
gggttagggttagggttaggg

SEQ ID NO: 2 - DNA, 22 bp, synthetic construct
  source 1..22: mol_type = other DNA
agggttagggttagggttaggg

SEQ ID NO: 3 - DNA, 24 bp, synthetic construct
  source 1..24: mol_type = other DNA
attgggttagggttagggttaggg

SEQ ID NO: 4 - DNA, 21 bp, synthetic construct
  source 1..21: mol_type = other DNA
gggttagggtgtgggttaggg

SEQ ID NO: 5 - DNA, 21 bp, synthetic construct
  source 1..21: mol_type = other DNA
gggttagggtctgggttaggg

SEQ ID NO: 6 - DNA, 20 bp, synthetic construct
  source 1..20: mol_type = other DNA
aggttttggcagggttttgg

SEQ ID NO: 7 - DNA, 19 bp, synthetic construct
  source 1..19: mol_type = other DNA
agggttgggtgtgggttgg

SEQ ID NO: 8 - DNA, 44 bp, synthetic construct
  source 1..44: mol_type = other DNA
tatttcttattcatattcaggaagacatcgagaccagggcacgc

SEQ ID NO: 9 - DNA, 52 bp, synthetic construct
  source 1..52: mol_type = other DNA
tatttcttattcatattcaggaagattttttttcatcgagaccagggcacgc

SEQ ID NO: 10 - DNA, 37 bp, synthetic construct
  source 1..37: mol_type = other DNA
attgggattgggtcttcctgaatatgaataagaaata

SEQ ID NO: 11 - DNA, 37 bp, synthetic construct
  source 1..37: mol_type = other DNA
tctgggattgggtcttcctgaatatgaataagaaata

SEQ ID NO: 12 - DNA, 28 bp, synthetic construct
  source 1..28: mol_type = other DNA
gcgtgccctggtctcgatggggttaggg

SEQ ID NO: 13 - DNA, 41 bp, synthetic construct
  source 1..41: mol_type = other DNA
agggattgggattgggtcttcctgaatatgaataagaaata

SEQ ID NO: 14 - DNA, 41 bp, synthetic construct
  source 1..41: mol_type = other DNA
agggtctgggattgggtcttcctgaatatgaataagaaata

SEQ ID NO: 15 - DNA, 24 bp, synthetic construct
  source 1..24: mol_type = other DNA
gcgtgccctggtctcgatggggtt

SEQ ID NO: 16 - DNA, 41 bp, synthetic construct
  source 1..41: mol_type = other DNA
atttatttttattttttcttcctgaatatgaataagaaata

SEQ ID NO: 17 - DNA, 41 bp, synthetic construct
  source 1..41: mol_type = other DNA
attttcttttattttttcttcctgaatatgaataagaaata

SEQ ID NO: 18 - DNA, 24 bp, synthetic construct
  source 1..24: mol_type = other DNA
gcgtgccctggtctcgatgttttt

SEQ ID NO: 19 - DNA, 20 bp, synthetic construct
  source 1..20: mol_type = other DNA
gccaggagaatgaggtggtc

SEQ ID NO: 20 - DNA, 37 bp, synthetic construct
  source 1..37: mol_type = other DNA
ttagggttagggtcttcctgaatatgaataagaaata

SEQ ID NO: 21 - DNA, 37 bp, synthetic construct
  source 1..37: mol_type = other DNA
tctgggttagggtcttcctgaatatgaataagaaata

SEQ ID NO: 22 - DNA, 40 bp, synthetic construct
  source 1..40: mol_type = other DNA
gggttagggttagggtcttcctgaatatgaataagaaata

SEQ ID NO: 23 - DNA, 40 bp, synthetic construct
  source 1..40: mol_type = other DNA
gggtctgggttagggtcttcctgaatatgaataagaaata

SEQ ID NO: 24 - DNA, 25 bp, synthetic construct
  source 1..25: mol_type = other DNA
gcgtgccctggtctcgatggggtta

SEQ ID NO: 25 - DNA, 21 bp, synthetic construct
  source 1..21: mol_type = other DNA
tatttcttattcatattcagg

SEQ ID NO: 26 - DNA, 22 bp, synthetic construct
  source 1..22: mol_type = other DNA
tatttcttattcatattcagga

SEQ ID NO: 27 - DNA, 24 bp, synthetic construct
  source 1..24: mol_type = other DNA
tatttcttattcatattcaggatt

SEQ ID NO: 28 - DNA, 29 bp, synthetic construct
  source 1..29: mol_type = other DNA
gcgtgccctggtctcgatgtctggggtta

SEQ ID NO: 29 - DNA, 43 bp, synthetic construct
  source 1..43: mol_type = other DNA
gggttagggttagggatgtcttcctgaatatgaataagaaata

SEQ ID NO: 30 - DNA, 17 bp, synthetic construct
  source 1..17: mol_type = other DNA
gggtagggcgggttggg

SEQ ID NO: 31 - DNA, 18 bp, synthetic construct
  source 1..18: mol_type = other DNA
gggtagggcgggttggga

SEQ ID NO: 32 - DNA, 20 bp, synthetic construct
  source 1..20: mol_type = other DNA
gggtagggcgggttgggaaa

SEQ ID NO: 33 - DNA, 18 bp, synthetic construct
  source 1..18: mol_type = other DNA
gggtagggcgggttaggg

SEQ ID NO: 34 - DNA, 17 bp, synthetic construct
  source 1..17: mol_type = other DNA
gggtagggcgggaaggg

SEQ ID NO: 35 - DNA, 41 bp, unidentified
  source 1..41: mol_type = other DNA
  misc_difference 33replace
  misc_difference 6replace
  misc_difference 24replace
  misc_difference 15replace
  misc_difference 1..5: note = 1, 2, 3, 4 or all 5 of the nucleotides can be absent
  misc_difference 28..32: note = 1, 2, 3, 4 or all 5 of the nucleotides can be absent
  misc_difference 19..23: note = 1, 2, 3, 4 or all 5 of the nucleotides can be absent
  misc_difference 10..14: note = 1, 2, 3, 4 or all 5 of the nucleotides can be absent
  misc_difference 37..41: note = 1, 2, 3, 4 or all 5 of the nucleotides can be absent
nnnnnggggnnnnnggggnnnnnggggnnnnnggggnnnnn

SEQ ID NO: 36 - DNA, 22 bp, synthetic construct
  source 1..22: mol_type = other DNA
agggttagggttagggttaggg

SEQ ID NO: 37 - DNA, 20 bp, synthetic construct
  source 1..20: mol_type = other DNA
tgggttgggttgggttgggt

SEQ ID NO: 38 - DNA, 23 bp, synthetic construct
  source 1..23: mol_type = other DNA
agggttagggttagggttaggga

SEQ ID NO: 39 - DNA, 25 bp, synthetic construct
  source 1..25: mol_type = other DNA
agggttagggttagggttagggaaa

SEQ ID NO: 40 - DNA, 23 bp, synthetic construct
  source 1..23: mol_type = other DNA
agggttagggttagggttaaggg

SEQ ID NO: 41 - DNA, 21 bp, synthetic construct
  source 1..21: mol_type = other DNA
tgggttgggttgggttgggta

SEQ ID NO: 42 - DNA, 23 bp, synthetic construct
  source 1..23: mol_type = other DNA
tgggttgggttgggttgggtaaa

SEQ ID NO: 43 - DNA, 21 bp, synthetic construct
  source 1..21: mol_type = other DNA
tgggttgggttgggttagggt

SEQ ID NO: 44 - DNA, 22 bp, synthetic construct
  source 1..22: mol_type = other DNA
tgggttgggttgggttaagggt

(19) United States
(12) Patent Application Publication
(10) Pub. No.: US 2026/0176609 A1
(43) Pub. Date: Jun. 25, 2026

(54) DNA CATALYST COMPOSITIONS AND METHODS OF DETECTION USING SAME

(71) Applicant: NORTHERN ILLINOIS RESEARCH FOUNDATION, Dekalb, IL (US)

(72) Inventors: Irina V. NESTROVA, Dekalb, IL (US); Elzbieta E. IWANIUK, Dekalb, IL (US)

(21) Appl. No.: 19/125,834

(22) PCT Filed: Nov. 3, 2023
(86) PCT No.: PCT/US2023/078605

§ 371 Date: Apr. 30, 2025

Related U.S. Application Data
(60) Provisional application No. 63/422,216, filed on Nov. 3, 2022.

(51) Int. Cl. C12N 9/96; C07H 21/04; C12N 15/113; C12Q 1/68; C12Q 1/6837

(52) U.S. Cl. CPC C12N 9/96; C07H 21/04; C12N 15/113; C12Q 1/68; C12Q 1/6837

(57) ABSTRACT

The present disclosure provides methods to detect a target in a sample by using a DNA scaffold with enzymatic activity (“DNAzyme”) that catalyzes hydrogen peroxidase decomposition, leading to a reaction wherein bubbles are produced. In particular, the present disclosure describes the capabilities of G-quadruplex·hemin constructs to produce bubbles as a signal transduction tool in biomarker detection. Observing bubbles is a straightforward and easy-to-interpret route towards instrument-free visualization of readouts that does not require a scientific background, an equipped lab, or a color vision proficiency.

### CROSS-REFERENCE TO RELATED APPLICATIONS

This application claims the benefit under 35 USC § 119 (e) of U.S. Provisional Application Ser. No. 63/422,216, filed on Nov. 3, 2022, the entire disclosure of which is incorporated herein by reference.

### GOVERNMENT SUPPORT STATEMENT

This invention was made with government support under grant number 1R15GM135796-01, awarded by The National Institutes of Health (NIH). The government has certain rights in the invention.

### REFERENCE TO AN ELECTRONIC SEQUENCE LISTING

The contents of the electronic sequence listing (file name: 396769.xml; size: 43 kilobytes, created on Nov. 2, 2023) is herein incorporated by reference in its entirety.

### BACKGROUND

Recently, artificial DNA scaffolds with enzymatic activity (“DNAzymes”) have emerged as signal transduction tools for use in chemical and biochemical analyses. These intrinsically stable, non-toxic, and affordable, small nucleic acid scaffolds align with requirements set forth by the World Health Organization (WHO) to operate under environments with limited resources, also known as the “ASSURED” (Affordable, Sensitive, Specific, User-Friendly, Robust and rapid, Equipment free, Deliverable) criteria.

One class of DNAzymes are G-quadruplexes·hemin constructs are valuable assets in the chemical/biochemical analysis field as translators of target recognition events into separation free readout (ASSURED-compliant. In particular, G-quadruplexes·hemin constructs mimic, but differ from, protein peroxidases. The constructs are compatible with readouts of different disciplines (e.g. optical, electrochemical and others). Discovery of oxidative catalysis with G-quadruplex·hemin constructs prompted a range of developments in the field of biosensor design. Thus, G-quadruplex-based DNAzymes with peroxidase activity have found a niche as signal transduction modules in a wide range of analytical applications.

A colorimetric readout is the only currently available equipment-free platform for transducing molecular recognition events and is used for the design of ASSURED-compliant molecular diagnostic devices to detect biomarkers (e.g., oligonucleotides). However, a disadvantage of diagnosis via colorimetric readout is that an interpretation of color intensity and hue is contingent on lighting conditions and/or on the color vision proficiency of the observer. In addition, it is difficult to detect color change in thin films. Therefore, the demand for equipment-free signal readouts of molecular detection events is unresolved and remains high.

### SUMMARY

A catalase is an enzyme that catalyzes hydrogen peroxide decomposition. Catalase activity of G-quadruplex·hemin constructs was established and its utility as a signal transduction platform was established in biomolecular recognition events by a proof-of-concept example of an oligonucleotide target.

Peroxidases require additional use of substrates in contrast to catalases wherein a G-quadruplex hemin system itself allows obtaining a signal. As used herein, a peroxidase refers to an enzyme that catalyzes oxidation by peroxides.

DNAzymes (G-quadruplex·hemin constructs) are able to catalyze hydrogen peroxide (H₂O₂) to form bubbles of molecular oxygen. Advantageously, these bubbles of oxygen are observable without special equipment in the presence of a target, for example an oligonucleotide, an antibody, or a protein. To enable biomarker detection, the constructs are capable of switching their conformation to the “active” topology of DNAzymes. The bubbles are released only when the target is present.

The new catalyst is activated upon conformational change. The conformational change is interfaced with detecting an intended molecular target, e.g., a diagnostic marker.

The present disclosure provides the capabilities of G-quadruplex·hemin constructs to produce bubbles as a signal transduction tool in biomarker detection, thus expanding upon previously reported structures known to have a peroxidase activity (which are different from that of a catalase). Catalase activity of hemin alone is negligible at ambient conditions because no oxygen bubbles are produced and thus cannot be observed. In the presence of an assembled G-quadruplex, however, catalytic activity of hemin is substantially enhanced yielding production of oxygen in the form of observable bubbles. Observing bubbles is a straightforward and easy-to-interpret route towards instrument-free visualization of readouts that does not require a scientific background, an equipped lab, or a color vision proficiency. Therefore, the methods disclosed herein are easily expandable to wide scale applications and is ASSURED compliant.

### BRIEF DESCRIPTION OF THE DRAWINGS

FIG. 1 shows a simplified catalytic cycles of peroxidases and catalases. “RS”=reducing substrate. Iron is in a Fe (III) state in resting hemin (upper structure in the center) and in a Fe (IV) state is in compound I and compound II.

FIGS. 2A-2E show bathochromic shift and increase in intensity of hemin's Soret band indicates hemin's de-aggregation upon interacting with G-quadruplex V, 0-75 min (FIG. 2A). Data for other quadruplexes indicate a similar trend: FIG. 2B: GI, 0-60 min; FIG. 2C: GII, 0-60 min; FIG. 2D: GIII, 0-60 min; FIG. 2E: GIV, 0-60 min; FIG. 2F: GVI, 0-50 min; FIG. 2G: GVII, 0-50 showing GVII. Hemin concentration is 500 μM in PBS buffer at pH 7.50 for all.

FIG. 3A shows Representative images and (B) summary of bubbles formation in presence of quadruplexes GI-GVII. Samples were prepared in PBS (pH 7.5) and contained equilibrated G-quadruplexes at indicated levels, hemin at 1 μM, and hydrogen peroxide at 29.3%. Controls consisted of all the reaction components except a G-quadruplex. Images in (A) are views on 20-mL glass vials from above taken with a cell phone camera. Observations in (B) are taken 30 minutes after adding hydrogen peroxide. One “bubble” symbol corresponds to 1-3 bubbles observed, two symbols to 4-8 bubbles, three to 8-12 bubbles, four to 12-18 bubbles, five to almost all the surface area covered (more than 18), a dash corresponds to zero (0) bubbles, that is, no target is detected.

FIG. 4A shows activation of catalase activity of quadruplexes GVIII and GIX in the presence of an oligonucleotide target requires splitting guanine-rich sequences in two parts. Two different split patterns were evaluated: a symmetric split (6:6) (FIG. 4A1) and an asymmetric split (9:3) (FIG. 4A2). FIG. 4B shows results over different split and target configurations. Each sample in PBS buffer (pH 7.50) including two quadruplex “arms” (R and L) and target (all at 500 nM concentrations), hemin at 1 μM, and hydrogen peroxide at 29.3%. Controls consisted of all the components except an oligonucleotide target. Observations were taken 30 minutes after adding hydrogen peroxide. One “bubble” symbol in the table corresponds to 1-3 bubbles observed, two symbols, to 4-8 bubbles, three to 8-12 bubbles. FIG. 4C shows a representative image of response of GVIII, 9:3 to the target T1 (left) and T2 (right) against a control (no target, center) taken with a 4× objective using a Keyance microscope. The samples were prepared in a 96-well plate. The total sample volume was 300 μL. FIG. 4D shows a schematics of catalase activation for antibody binding. When two probes (P) are bound to antibodies (red T), the quadruplex parts (small blue circles) will combine to form a G-quadruplex enzyme (TPP). FIG. 4E shows the catalase activation principle can be extended to proteins with two peptide or oligonucleotide binding sites. The split catalase fragments (blue) will be fused to the binding fragments (red). They will recombine to activate catalase only upon binding to target.

FIGS. 5A-5D show a top view on 20-ml glass vials of a catalase reaction activated by quadruplexes. FIG. 5A shows GV and control without quadruplex (second row). FIG. 5B shows GVI. FIG. 5C shows GVII. FIG. 5D shows zoomed in frames i-iv (bottom row) as labeled. The reaction was performed in PBS buffer at pH 7.5 in presence of 1 μM hemin and 29.7% H₂O₂. Oligonucleotides GV, GVI, and GVII were at 200 nM.

FIG. 6 shows 20% PAGE electrophoresis in 0.5×TBE indicates that all full quadruplexes (GI-GVII) are folded in PBS pH 7.5 buffer. Every quadruplex (at ˜5 μM concentration) was injected in duplicate. The quadruplex injections were intermitted with injections of a 20-nt single stranded oligonucleotide deliberately designed not to form secondary intramolecular folds. To visualize oligonucleotides, the gels were stained in SYBR Gold (3 μL dye in 200 mL of 0.5×TBE buffer).

FIG. 7A-7H show the CD spectra of full quadruplexes GI-GVIII and the quadruplex system GVIII (9:3 split (7H)). All oligonucleotide sequences are provided in Table 1. The measurements were performed in PBS buffer (pH 7.5); oligonucleotide concentrations were ˜5 μM.

FIG. 8 shows probes (P) for quantitative analysis are designed in such a way that although a target (T) can bind both probes, the binding of each individual probe decreases binding affinity of another probe (due to binding region overlap, blue shaded box). The example illustrates probe design (TPP) for oligonucleotide targets.

FIG. 9 shows a schematic for a potential ASSURED-compliant molecular diagnostic system. The testing system will present a kit can include a sample swab, a tube with pre-mixed reagents for biomarker extraction and a pipette with catalase and probes. First, the sample will be exposed to the extraction buffer. Next, the catalase and probes will be added. After an appropriate time, the tube will be examined for the presence of bubbles. Bubble appearance will indicate a biomarker presence. Absence of bubbles will indicate no biomarker.

FIG. 10 shows an intact quadruplex modified with domain C and synthesized hemin-modified Domain D through EDC/NHS coupling.

FIG. 11 shows that the target-assembly activatable system (split) is compatible with a stoichiometric approach for quantitative analysis of biomolecules in that the approach enables naked eye quantitative analysis of a target T1 at 500 nM level.

FIG. 12 shows catalase activity of quadruplex C2 using PBS buffer, pH 7.5, H₂O₂. Images of top view of scintillation vials are taken with a cell phone camera.

FIG. 13 shows the experimental approach for identifying core quadruplexes and establishing quadruplex structure-catalase activity correlations.

FIG. 14 shows structures of reagents for modifying oligonucleotides with imidazole.

FIG. 15 shows a split quadruplex strategy.

FIG. 16 shows various approaches to quadruplex splitting.

FIG. 17 demonstrates the symmetrically split quadruplex GGG TTA GGG TCT GGG TTA GGG (SEQ ID NO: 5) shows zero background (25 min, “no target”) but the number of bubbles upon target addition (25 min, “with target”) is rather small.

FIG. 18 shows the melting profile of quadruplex GGG TTA GGG TCT GGG TTA GGG (SEQ ID NO: 5), a parent quadruplex to a symmetric split from FIG. 17.

FIG. 19 shows design of protective hairpins. The hairpins can vary in strength (through the number of base pairs in stem and GC/content) and by protected region within guanine stretches.

FIG. 20 shows a signaling scheme for antibodies based on symmetrically split quadruplexes.

FIG. 21 shows a model system for linker selection. Rigid double stranded duplex can position split parts of quadruplex at a distance similar to antibody-bound. The linker length and flexibility will be adjusted.

### DETAILED DISCLOSURE

Direct indications of a catalase activity by nucleic acid scaffolds have not been previously reported. Prior to the present disclosure, activatable G-quadruplex·hemin constructs have not been used to produce bubbles (in general) nor was it suggested to activate catalase activity of hemin as a mechanism to visualize molecular diagnostics. An activatable catalase refers to a catalase that becomes active only in presence of a target.

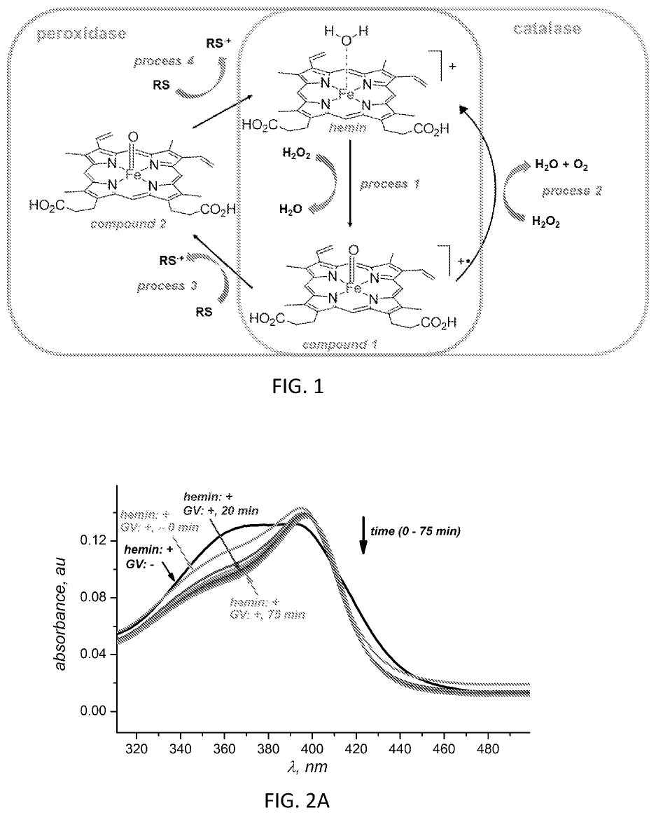

The present disclosure demonstrates that G-quadruplex·hemin constructs can function as catalases. In the field of proteins, both heme-peroxidases and catalases belong to the same class of metalloenzymes that operate from catalytic activity of heme, and their catalytic cycles are similar (see, e.g., a simplified catalytic cycle shown in FIG. 1). Thus, a required step in both enzymatic pathways is the formation of Compound I (FIG. 1, process 1).

In contrast, for catalases, Compound I reacts with a second molecule of H₂O₂ yielding O₂, H₂O, and the regenerated enzyme (FIG. 1, process 2). In peroxidases, Compound I reacts with a reducing substrate (RS) to form Compound II (FIG. 1, process 3) followed by regenerating a resting enzyme (FIG. 1, process 4).

However, the signal output in case of catalases principally differs because catalases produce bubbles, while peroxidases change color. The bubbles produced by catalases can be used to visualize molecular diagnostics.

In an illustrative aspect, a method to detect a target in a sample is provided. The method comprises the steps of a) obtaining a DNAzyme that catalyzes hydrogen peroxidase decomposition; and b) contacting the DNAzyme to the sample to produce a reaction, wherein bubbles are produced via the reaction when the target is present in the sample.

In an embodiment, the target is selected from a group consisting of a biomarker, an oligonucleotide, an antibody, a peptide and a small molecule. In an embodiment, the target is a biomarker. As used herein a biomarker refers to a molecule of which the presence, absence, or quantity is an indicator of a normal biological process, a pathogenic process, or a response to a therapeutic intervention.

In an embodiment, the target is an oligonucleotide. In an embodiment, the target is an antibody. In an embodiment, the target is a peptide. In an embodiment, the target is a small molecule.

In an embodiment, the sample is a biological sample. In an embodiment, the biological sample is saliva. In an embodiment, the biological sample is blood. In an embodiment, the biological sample is plasma. In an embodiment, the biological sample is mucus.

In an embodiment, the DNAzyme is a G-quadruplex·hemin. In an embodiment, the G-quadruplex comprises an oligonucleotide. In an embodiment, the oligonucleotide comprises a sequence comprising NₐG_{b}NₐG_{b}NₐG_{b}NₐG_{b}Nₐ (SEQ ID NO: 35), wherein G represents guanine, wherein N represents one of guanine (G), thymidine (T), adenine (A), cytosine (C), wherein each a in the sequence, independently, is a number between 0 and 5, and wherein each b in the sequence, independently, is either 3 or 4. In an embodiment, N represents guanine (G). In an embodiment, N represents thymidine (T). In an embodiment, N represents adenine (A). In an embodiment, N represents cytosine (C). In an embodiment, a is a number that equals 0. In an embodiment, a is a number that equals 1. In an embodiment, a is a number that equals 2. In an embodiment, a is a number that equals 3. In an embodiment, a is a number that equals 4. In an embodiment, a is a number that equals 5. It is contemplated that for each a in the sequence, independently, can be a number between 0 and 5 (e.g., in the sequence comprising NₐG_{b}NₐG_{b}NₐG_{b}NₐG_{b}Nₐ (SEQ ID NO: 35), the first a could be 1, the second a could be 3, the third a can be 4, and the like).

In an embodiment, b is a number that equals 3. In an embodiment, b is a number that equals 4. It is contemplated that for each b in the sequence, independently, can be either 3 or 4 (e.g., in the sequence comprising NₐG_{b}NₐG_{b}NₐG_{b}NₐG_{b}Nₐ (SEQ ID NO: 35), the first b could be 4, the second b could be 3, the third b can be 4, and the like).

In an embodiment, the oligonucleotide comprises SEQ ID NO: 1. In an embodiment, the oligonucleotide comprises SEQ ID NO: 2. In an embodiment, the oligonucleotide comprises SEQ ID NO: 3. In an embodiment, the oligonucleotide comprises SEQ ID NO: 4. In an embodiment, the oligonucleotide comprises SEQ ID NO: 5. In an embodiment, the oligonucleotide comprises SEQ ID NO: 6. In an embodiment, the oligonucleotide comprises SEQ ID NO: 7. In an embodiment, the oligonucleotide comprises SEQ ID NO: 8. In an embodiment, the oligonucleotide comprises SEQ ID NO: 9. In an embodiment, the oligonucleotide comprises SEQ ID NO: 10. In an embodiment, the oligonucleotide comprises SEQ ID NO: 11. In an embodiment, the oligonucleotide comprises SEQ ID NO: 12. In an embodiment, the oligonucleotide comprises SEQ ID NO: 13. In an embodiment, the oligonucleotide comprises SEQ ID NO: 14. In an embodiment, the oligonucleotide comprises SEQ ID NO: 15. In an embodiment, the oligonucleotide comprises SEQ ID NO: 16. In an embodiment, the oligonucleotide comprises SEQ ID NO: 17. In an embodiment, the oligonucleotide comprises SEQ ID NO: 18. In an embodiment, the oligonucleotide comprises SEQ ID NO: 20. In an embodiment, the oligonucleotide comprises SEQ ID NO: 21. In an embodiment, the oligonucleotide comprises SEQ ID NO: 22. In an embodiment, the oligonucleotide comprises SEQ ID NO: 23. In an embodiment, the oligonucleotide comprises SEQ ID NO: 24. In an embodiment, the oligonucleotide comprises SEQ ID NO: 28. In an embodiment, the oligonucleotide comprises SEQ ID NO: 29.

In an embodiment, the oligonucleotide comprises a confirmation in a folded state. In an embodiment, the oligonucleotide comprises a confirmation in an unfolded state.

In an embodiment, the reaction comprises decomposition of hydrogen peroxide. In an embodiment, the bubbles comprise O₂.

In an embodiment, the method is performed at a pH between 7 and 11. In an embodiment, the method is performed at a pH between 7 and 8. In an embodiment, the method is performed at a pH between 8 and 9. In an embodiment, the method is performed at a pH between 9 and 10. In an embodiment, the method is performed at a pH between 10 and 11. In an embodiment, the method is performed at a pH of 7. In an embodiment, the method is performed at a pH of 7.5. In an embodiment, the method is performed at a pH of 8. In an embodiment, the method is performed at a pH of 8.5. In an embodiment, the method is performed at a pH of 9. In an embodiment, the method is performed at a pH of 9.5. In an embodiment, the method is performed at a pH of 10. In an embodiment, the method is performed at a pH of 10.5. In an embodiment, the method is performed at a pH of 11.

In an illustrative aspect, a kit for detection of a target in a sample is provided. The kit comprises a swab for the sample, a DNAzyme that catalyzes hydrogen peroxidase decomposition, and a container for reacting the sample with the DNAzyme, wherein the sample and the DNAzyme are capable of being contacted in the container to form bubbles.

In an embodiment, the target is selected from a group consisting of a biomarker, an oligonucleotide, an antibody, a peptide and a small molecule. In an embodiment, the target is a biomarker. In an embodiment, the target is an oligonucleotide. In an embodiment, the target is an antibody. In an embodiment, the target is a peptide. In an embodiment, the target is a small molecule.

In an embodiment, the sample is a biological sample. In an embodiment, the biological sample is saliva. In an embodiment, the biological sample is blood. In an embodiment, the biological sample is plasma. In an embodiment, the biological sample is mucus.

In an embodiment, the DNAzyme is a G-quadruplex·hemin. In an embodiment, the G-quadruplex comprises an oligonucleotide. In an embodiment, the oligonucleotide comprises a sequence comprising NₐG_{b}NₐG_{b}NₐG_{b}NₐG_{b}Nₐ (SEQ ID NO: 35), wherein G represents guanine, wherein N represents one of guanine (G), thymidine (T), adenine (A), cytosine (C), wherein each a in the sequence, independently, is a number between 0 and 5, and wherein each b in the sequence, independently, is either 3 or 4.

In an embodiment, the oligonucleotide comprises SEQ ID NO: 1. In an embodiment, the oligonucleotide comprises SEQ ID NO: 2. In an embodiment, the oligonucleotide comprises SEQ ID NO: 3. In an embodiment, the oligonucleotide comprises SEQ ID NO: 4. In an embodiment, the oligonucleotide comprises SEQ ID NO: 5. In an embodiment, the oligonucleotide comprises SEQ ID NO: 6. In an embodiment, the oligonucleotide comprises SEQ ID NO: 7. In an embodiment, the oligonucleotide comprises SEQ ID NO: 8. In an embodiment, the oligonucleotide comprises SEQ ID NO: 9. In an embodiment, the oligonucleotide comprises SEQ ID NO: 10. In an embodiment, the oligonucleotide comprises SEQ ID NO: 11. In an embodiment, the oligonucleotide comprises SEQ ID NO: 12. In an embodiment, the oligonucleotide comprises SEQ ID NO: 13. In an embodiment, the oligonucleotide comprises SEQ ID NO: 14. In an embodiment, the oligonucleotide comprises SEQ ID NO: 15. In an embodiment, the oligonucleotide comprises SEQ ID NO: 16. In an embodiment, the oligonucleotide comprises SEQ ID NO: 17. In an embodiment, the oligonucleotide comprises SEQ ID NO: 18. In an embodiment, the oligonucleotide comprises SEQ ID NO: 20. In an embodiment, the oligonucleotide comprises SEQ ID NO: 21. In an embodiment, the oligonucleotide comprises SEQ ID NO: 22. In an embodiment, the oligonucleotide comprises SEQ ID NO: 23. In an embodiment, the oligonucleotide comprises SEQ ID NO: 24. In an embodiment, the oligonucleotide comprises SEQ ID NO: 28. In an embodiment, the oligonucleotide comprises SEQ ID NO: 29.

In an embodiment, the oligonucleotide comprises a confirmation in a folded state. In an embodiment, the oligonucleotide comprises a confirmation in an unfolded state.

In an embodiment, the kit is capable of providing a reaction that comprises decomposition of hydrogen peroxide. In an embodiment, the bubbles comprise O₂.

The following numbered embodiments are contemplated and are non-limiting:

1. A method to detect a target in a sample, the method comprising:
(a) obtaining a DNAzyme that catalyzes hydrogen peroxidase decomposition;
(b) contacting the DNAzyme to the sample to produce a reaction, wherein bubbles are produced via the reaction when the target is present in the sample.

2. The method of clause 1, any other suitable clause, or any combination of suitable clauses, wherein the target is selected from a group consisting of a biomarker, an oligonucleotide, an antibody, a peptide and a small molecule.

3. The method of clause 1, any other suitable clause, or any combination of suitable clauses, wherein the target is a biomarker.

4. The method of clause 1, any other suitable clause, or any combination of suitable clauses, wherein the target is an oligonucleotide.

5. The method of clause 1, any other suitable clause, or any combination of suitable clauses, wherein the target is an antibody.

6. The method of clause 1, any other suitable clause, or any combination of suitable clauses, wherein the target is a peptide.

7. The method of clause 1, any other suitable clause, or any combination of suitable clauses, wherein the target is a small molecule.

8. The method of clause 1, any other suitable clause, or any combination of suitable clauses, wherein the sample is a biological sample.

9. The method of clause 8, any other suitable clause, or any combination of suitable clauses, wherein the biological sample is saliva.

10. The method of clause 8, any other suitable clause, or any combination of suitable clauses, wherein the biological sample is blood.

11. The method of clause 8, any other suitable clause, or any combination of suitable clauses, wherein the biological sample is plasma.

12. The method of clause 8, any other suitable clause, or any combination of suitable clauses, wherein the biological sample is mucus.

13. The method of clause 1, any other suitable clause, or any combination of suitable clauses, wherein the DNAzyme is a G-quadruplex·hemin.

14. The method of clause 13, any other suitable clause, or any combination of suitable clauses, wherein the G-quadruplex comprises an oligonucleotide.

15. The method of clause 14, any other suitable clause, or any combination of suitable clauses, wherein the oligonucleotide comprises a sequence comprising NₐG_{b}NₐG_{b}NₐG_{b}NₐG_{b}Nₐ (SEQ ID NO: 35),
wherein G represents guanine,
wherein N represents one of guanine (G), thymidine (T), adenine (A), cytosine (C),
wherein each a in the sequence, independently, is a number between 0 and 5, and
wherein each b in the sequence, independently, is either 3 or 4.

16. The method of clause 14, any other suitable clause, or any combination of suitable clauses, wherein the oligonucleotide comprises SEQ ID NO: 1.

17. The method of clause 14, any other suitable clause, or any combination of suitable clauses, wherein the oligonucleotide comprises SEQ ID NO: 2.

18. The method of clause 14, any other suitable clause, or any combination of suitable clauses, wherein the oligonucleotide comprises SEQ ID NO: 3.

19. The method of clause 14, any other suitable clause, or any combination of suitable clauses, wherein the oligonucleotide comprises SEQ ID NO: 4.

20. The method of clause 14, any other suitable clause, or any combination of suitable clauses, wherein the oligonucleotide comprises SEQ ID NO: 5.

21. The method of clause 14, any other suitable clause, or any combination of suitable clauses, wherein the oligonucleotide comprises SEQ ID NO: 6.

22. The method of clause 14, any other suitable clause, or any combination of suitable clauses, wherein the oligonucleotide comprises SEQ ID NO: 7.

23. The method of clause 14, any other suitable clause, or any combination of suitable clauses, wherein the oligonucleotide comprises SEQ ID NO: 8.

24. The method of clause 14, any other suitable clause, or any combination of suitable clauses, wherein the oligonucleotide comprises SEQ ID NO: 9.

25. The method of clause 14, any other suitable clause, or any combination of suitable clauses, wherein the oligonucleotide comprises SEQ ID NO: 10.

26. The method of clause 14, any other suitable clause, or any combination of suitable clauses, wherein the oligonucleotide comprises SEQ ID NO: 11.

27. The method of clause 14, any other suitable clause, or any combination of suitable clauses, wherein the oligonucleotide comprises SEQ ID NO: 12.

28. The method of clause 14, any other suitable clause, or any combination of suitable clauses, wherein the oligonucleotide comprises SEQ ID NO: 13.

29. The method of clause 14, any other suitable clause, or any combination of suitable clauses, wherein the oligonucleotide comprises SEQ ID NO: 14.

30. The method of clause 14, any other suitable clause, or any combination of suitable clauses, wherein the oligonucleotide comprises SEQ ID NO: 15.

31. The method of clause 14, any other suitable clause, or any combination of suitable clauses, wherein the oligonucleotide comprises SEQ ID NO: 16.

32. The method of clause 14, any other suitable clause, or any combination of suitable clauses, wherein the oligonucleotide comprises SEQ ID NO: 17.

33. The method of clause 14, any other suitable clause, or any combination of suitable clauses, wherein the oligonucleotide comprises SEQ ID NO: 18.

34. The method of clause 14, any other suitable clause, or any combination of suitable clauses, wherein the oligonucleotide comprises SEQ ID NO: 20.

35. The method of clause 14, any other suitable clause, or any combination of suitable clauses, wherein the oligonucleotide comprises SEQ ID NO: 21.

36. The method of clause 14, any other suitable clause, or any combination of suitable clauses, wherein the oligonucleotide comprises SEQ ID NO: 22.

37. The method of clause 14, any other suitable clause, or any combination of suitable clauses, wherein the oligonucleotide comprises SEQ ID NO: 23.

38. The method of clause 14, any other suitable clause, or any combination of suitable clauses, wherein the oligonucleotide comprises SEQ ID NO: 24.

39. The method of clause 14, any other suitable clause, or any combination of suitable clauses, wherein the oligonucleotide comprises SEQ ID NO: 28.

40. The method of clause 14, any other suitable clause, or any combination of suitable clauses, wherein the oligonucleotide comprises SEQ ID NO: 29.

41. The method of clause 14, any other suitable clause, or any combination of suitable clauses, wherein the oligonucleotide comprises a confirmation in a folded state.

42. The method of clause 14, any other suitable clause, or any combination of suitable clauses, wherein the oligonucleotide comprises a confirmation in an unfolded state.

43. The method of clause 1, any other suitable clause, or any combination of suitable clauses, wherein the reaction comprises decomposition of hydrogen peroxide.

44. The method of clause 1, any other suitable clause, or any combination of suitable clauses, wherein the bubbles comprise O₂.

45. The method of clause 1, any other suitable clause, or any combination of suitable clauses, wherein the method is performed at a pH between 7 and 11.

46. The method of clause 1, any other suitable clause, or any combination of suitable clauses, wherein the method is performed at a pH between 7 and 8.

47. The method of clause 1, any other suitable clause, or any combination of suitable clauses, wherein the method is performed at a pH between 8 and 9.

48. The method of clause 1, any other suitable clause, or any combination of suitable clauses, wherein the method is performed at a pH between 9 and 10.

49. The method of clause 1, any other suitable clause, or any combination of suitable clauses, wherein the method is performed at a pH between 10 and 11.

50. The method of clause 1, any other suitable clause, or any combination of suitable clauses, wherein the method is performed at a pH of 7.

51. The method of clause 1, any other suitable clause, or any combination of suitable clauses, wherein the method is performed at a pH of 7.5.

52. The method of clause 1, any other suitable clause, or any combination of suitable clauses, wherein the method is performed at a pH of 8.

53. The method of clause 1, any other suitable clause, or any combination of suitable clauses, wherein the method is performed at a pH of 8.5.

54. The method of clause 1, any other suitable clause, or any combination of suitable clauses, wherein the method is performed at a pH of 9.

55. The method of clause 1, any other suitable clause, or any combination of suitable clauses, wherein the method is performed at a pH of 9.5.

56. The method of clause 1, any other suitable clause, or any combination of suitable clauses, wherein the method is performed at a pH of 10.

57. The method of clause 1, any other suitable clause, or any combination of suitable clauses, wherein the method is performed at a pH of 10.5.

58. The method of clause 1, any other suitable clause, or any combination of suitable clauses, wherein the method is performed at a pH of 11.

59. A kit for detection of a target in a sample, the kit comprising
a swab for the sample,
a DNAzyme that catalyzes hydrogen peroxidase decomposition, and
a container for reacting the sample with the DNAzyme, wherein the sample and the DNAzyme are capable of being contacted in the container to form bubbles.

60. The kit of clause 59, any other suitable clause, or any combination of suitable clauses, wherein the target is selected from a group consisting of a biomarker, an oligonucleotide, an antibody, a peptide and a small molecule.

61. The kit of clause 59, any other suitable clause, or any combination of suitable clauses, wherein the target is a biomarker.

62. The kit of clause 59, any other suitable clause, or any combination of suitable clauses, wherein the target is an oligonucleotide.

63. The kit of clause 59, any other suitable clause, or any combination of suitable clauses, wherein the target is an antibody.

64. The kit of clause 59, any other suitable clause, or any combination of suitable clauses, wherein the target is a peptide.

65. The kit of clause 59, any other suitable clause, or any combination of suitable clauses, wherein the target is a small molecule.

66. The kit of clause 59, any other suitable clause, or any combination of suitable clauses, wherein the sample is a biological sample.

67. The kit of clause 66, any other suitable clause, or any combination of suitable clauses, wherein the biological sample is saliva.

68. The kit of clause 66, any other suitable clause, or any combination of suitable clauses, wherein the biological sample is blood.

69. The kit of clause 66, any other suitable clause, or any combination of suitable clauses, wherein the biological sample is plasma.

70. The kit of clause 66, any other suitable clause, or any combination of suitable clauses, wherein the biological sample is mucus.

71. The kit of clause 59, any other suitable clause, or any combination of suitable clauses, wherein the DNAzyme is a G-quadruplex·hemin.

72. The kit of clause 71, any other suitable clause, or any combination of suitable clauses, wherein the G-quadruplex comprises an oligonucleotide.

73. The kit of clause 72, any other suitable clause, or any combination of suitable clauses, wherein the oligonucleotide comprises a sequence comprising NₐG_{b}NₐG_{b}NₐG_{b}NₐG_{b}Nₐ (SEQ ID NO: 35), wherein G represents guanine,
wherein N represents one of guanine (G), thymidine (T), adenine (A), cytosine (C),
wherein each a in the sequence, independently, is a number between 0 and 5, and
wherein each b in the sequence, independently, is either 3 or 4.

74. The kit of clause 72, any other suitable clause, or any combination of suitable clauses, wherein the oligonucleotide comprises SEQ ID NO: 1.

75. The kit of clause 72, any other suitable clause, or any combination of suitable clauses, wherein the oligonucleotide comprises SEQ ID NO: 2.

76. The kit of clause 72, any other suitable clause, or any combination of suitable clauses, wherein the oligonucleotide comprises SEQ ID NO: 3.

77. The kit of clause 72, any other suitable clause, or any combination of suitable clauses, wherein the oligonucleotide comprises SEQ ID NO: 4.

78. The kit of clause 72, any other suitable clause, or any combination of suitable clauses, wherein the oligonucleotide comprises SEQ ID NO: 5.

79. The kit of clause 72, any other suitable clause, or any combination of suitable clauses, wherein the oligonucleotide comprises SEQ ID NO: 6.

80. The kit of clause 72, any other suitable clause, or any combination of suitable clauses, wherein the oligonucleotide comprises SEQ ID NO: 7.

81. The kit of clause 72, any other suitable clause, or any combination of suitable clauses, wherein the oligonucleotide comprises SEQ ID NO: 8.

82. The kit of clause 72, any other suitable clause, or any combination of suitable clauses, wherein the oligonucleotide comprises SEQ ID NO: 9.

83. The kit of clause 72, any other suitable clause, or any combination of suitable clauses, wherein the oligonucleotide comprises SEQ ID NO: 10.

84. The kit of clause 72, any other suitable clause, or any combination of suitable clauses, wherein the oligonucleotide comprises SEQ ID NO: 11.

85. The kit of clause 72, any other suitable clause, or any combination of suitable clauses, wherein the oligonucleotide comprises SEQ ID NO: 12.

86. The kit of clause 72, any other suitable clause, or any combination of suitable clauses, wherein the oligonucleotide comprises SEQ ID NO: 13.

87. The kit of clause 72, any other suitable clause, or any combination of suitable clauses, wherein the oligonucleotide comprises SEQ ID NO: 14.

88. The kit of clause 72, any other suitable clause, or any combination of suitable clauses, wherein the oligonucleotide comprises SEQ ID NO: 15.

89. The kit of clause 72, any other suitable clause, or any combination of suitable clauses, wherein the oligonucleotide comprises SEQ ID NO: 16.

90. The kit of clause 72, any other suitable clause, or any combination of suitable clauses, wherein the oligonucleotide comprises SEQ ID NO: 17.

91. The kit of clause 72, any other suitable clause, or any combination of suitable clauses, wherein the oligonucleotide comprises SEQ ID NO: 18.

92. The kit of clause 72, any other suitable clause, or any combination of suitable clauses, wherein the oligonucleotide comprises SEQ ID NO: 20.

93. The kit of clause 72, any other suitable clause, or any combination of suitable clauses, wherein the oligonucleotide comprises SEQ ID NO: 21.

94. The kit of clause 72, any other suitable clause, or any combination of suitable clauses, wherein the oligonucleotide comprises SEQ ID NO: 22.

95. The kit of clause 72, any other suitable clause, or any combination of suitable clauses, wherein the oligonucleotide comprises SEQ ID NO: 23.

96. The kit of clause 72, any other suitable clause, or any combination of suitable clauses, wherein the oligonucleotide comprises SEQ ID NO: 24.

97. The kit of clause 72, any other suitable clause, or any combination of suitable clauses, wherein the oligonucleotide comprises SEQ ID NO: 28.

98. The kit of clause 72, any other suitable clause, or any combination of suitable clauses, wherein the oligonucleotide comprises SEQ ID NO: 29.

99. The kit of clause 72, any other suitable clause, or any combination of suitable clauses, wherein the oligonucleotide comprises a confirmation in a folded state.

100. The kit of clause 72, any other suitable clause, or any combination of suitable clauses, wherein the oligonucleotide comprises a confirmation in an unfolded state.

101. A method to quantitate a target in a sample, the method comprising:
(a) contacting the sample to a plurality of probes, wherein the target is capable of binding to more than one probe, wherein binding of the target to an individual probe results in a decreased binding affinity to remaining probes in the plurality of probes;
(b) exposing the sample to varying concentrations of the plurality of probes, wherein the probes bind to the target in the sample to provide one or more target-probe hybrids; and
(c) analyzing the target-probe hybrids to quantify the amount of the target in the sample based on the binding of the target to the varying concentrations of the plurality of probes.

### EXAMPLE 1

### Materials and Methods

The instant example provides materials and methods that were utilized in Examples 2-4.

### Materials

Chemical reagents were obtained from established commercial suppliers (e.g. Sigma-Aldrich (St. Louis, MO), Fisher Scientific (Pittsburgh, PA) etc.) unless specifically mentioned below. Nuclease-free water used for preparation of DNA solutions was obtained from IDT (Coralville, IA). 10×PBS Buffer (pH 7.4) was purchased from Thermo Fisher Scientific (Waltham, MA); UltraPure 1M Tris-HCl (pH 7.5) Buffer was purchased from Invitrogen (Grand Island, N.Y.) Single stranded DNAs were obtained from IDT (Coralville, IA) and reconstituted with nuclease-free water. Oligonucleotide sequences are included in Table 1. pH-meter calibration buffers (4.00±0.01, 7.00±0.01, and 10.00±0.02) were purchased from Fisher Scientific. DNA TriDye Ultra Low Range DNA Ladder was purchased from New England Biolabs (Ipswich, MA). SYBR Gold staining dye was purchased from Thermo Fisher Scientific. Acrylamide: bis-Acrylamide (29:1) solution for PAGE was obtained from BioRad (Hercules, CA). Hydrogen Peroxide (30%) was purchased from Fisher Scientific. Hemin was purchased from Thermo Fisher Scientific. TRIS buffers supplemented with NaCl and KCl were prepared according to general procedures.

### G-Quadruplex Preparation

G-quadruplexes were prepared by denaturing an approximately 150 μL of a 10-50 μM oligonucleotide solution in an appropriate buffer at 95.0° C. for 5.0 minutes followed by overnight cooling to a room temperature. The actual concentration of G-quadruplexes was established post-equilibration using 260 nm absorption values obtained by UV-vis and extinction coefficients provided by IDT.

### UV-Vis Measurements

Cary 4000 UV-Vis spectrophotometer (Agilent Technologies, Santa Clara, CA) was used for all UV measurements. Typically, measurements were performed on 800-μL solution aliquots using a 10 mm optical path quartz cuvette. For establishing oligonucleotide concentrations, ˜1 μM dilutions were analyzed.

### Measuring pH

ThermoOrion pH-meter (Model 420) was used throughout the studies. The pH meter was calibrated against fresh aliquots of pH calibration buffers before each use.

### Circular Dichroism (CD) Spectrometry

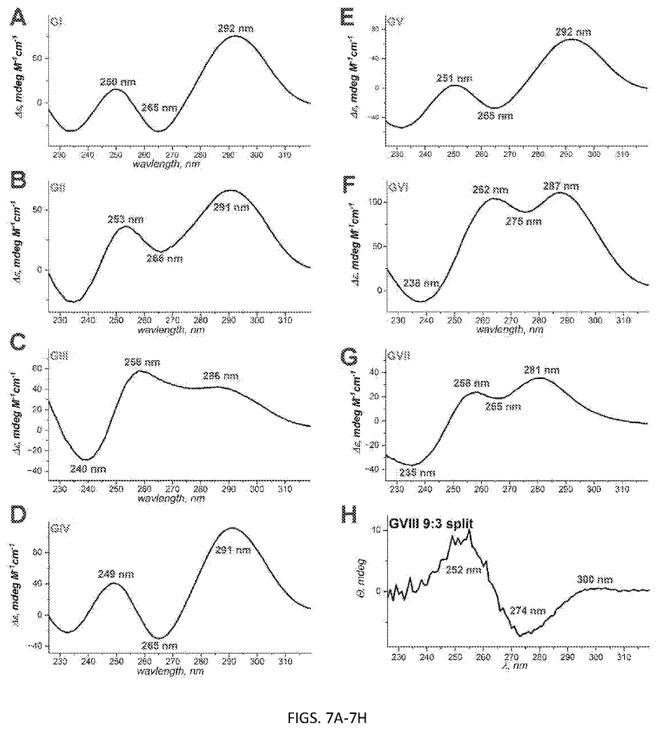

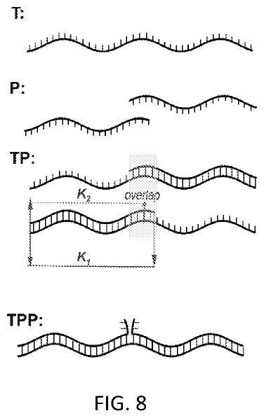

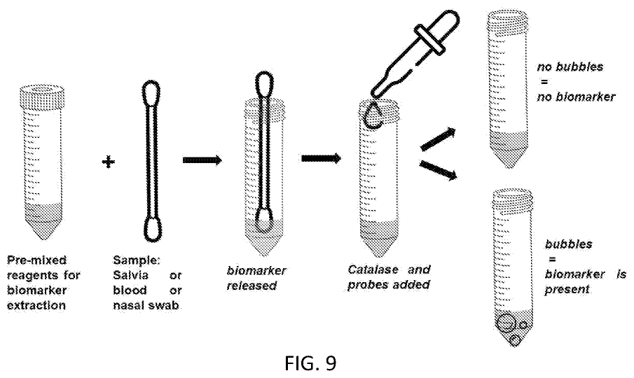

As shown in FIG. 7, CD spectra were collected on AVIV Model 215 Circular Dichroism Spectrometer (Aviv Instruments Inc., Lakewood, NJ) from 200 to 320 nm. Solutions containing equilibrated G-quadruplexes (prepared as described in G-quadruplex preparation subsection above) were diluted to ˜5 μM with PBS buffer at pH of 7.5. Quartz cuvettes with a 10-mm light path were used for all CD measurements. CD spectra for split structures were recorded in the presence of the equivalent amount of target T1. To correct for a signal from DNA duplex in target/G-quadruplex constructs, the control “duplex” (target T1+9:3 split T control L+9:3 split T control R) spectrum was subtracted from corresponding split G-quadruplexes+target T1 spectra.

### Gel Electrophoresis

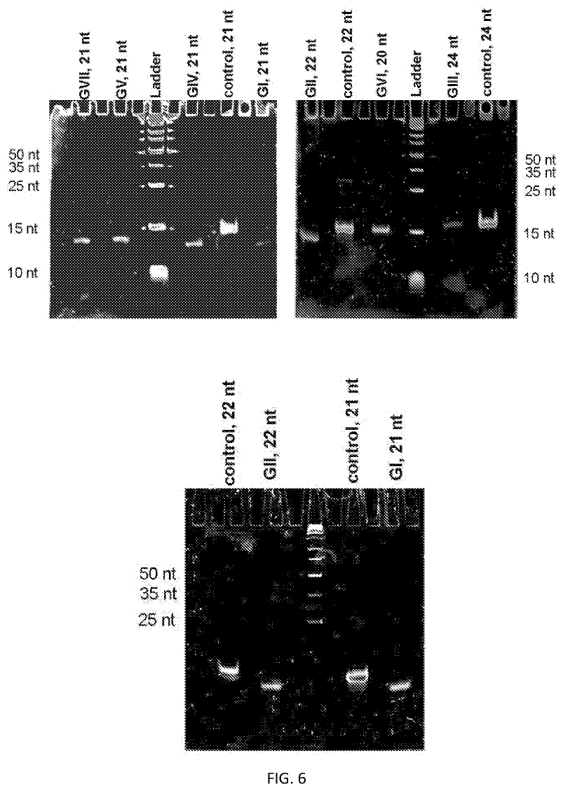

Gels for polyacrylamide gel electrophoresis (PAGE) were prepared according to general procedures. Typically, 6 μL of ˜5 μM oligonucleotide solutions were separated in 0.5×TBE running buffer on a 20% gel at 150 V for 1.5 hours using VWR (Radnor, PA) vertical PAGE system and power source. Gels were stained in SYBR Gold (3 μL dye in 200 mL of 0.5×TBE buffer) for 20 minutes and imaged with Bio-Rad EZ Gel Doc imager (Hercules, CA). A 20-nt single stranded oligonucleotide was injected as an unfolded control. Results are shown in FIG. 6.

### Hemin Solutions

A 2.8 mM hemin's stock was prepared by dissolving 9 mg of hemin in 5 mL of DMSO. A 500 μM working solution was diluted from the stock in DMSO.

### G-Quadruplex-Hemin Binding

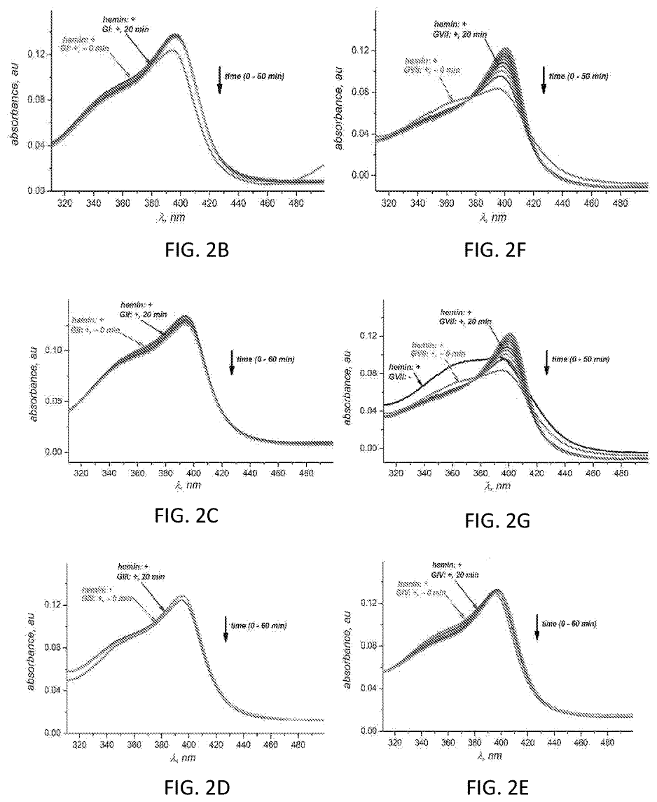

For each quadruplex GI-GVII, equilibrated oligonucleotide solutions were added to hemin in PBS (pH 7.5) to yield a final concentration of hemin at 1 μM and of an oligonucleotide at 500 nM. Absorption spectra from 300 nm to 500 nm were recorded every 5 minutes for 60-75 minutes. Results are shown in FIG. 2.

### Catalase Activity of Full G-Quadruplexes GI-GVII

For naked eye observations, 40 μL of equilibrated 10-50 μM G-quadruplex solution (described herein) was mixed with 4 μL of a hemin working solution (500 μM in DMSO) in a 20-mL glass scintillation vial. The mixture was equilibrated for 20 minutes at ambient conditions to let hemin interact with G-quadruplex. Then 1956 μL of 30% hydrogen peroxide was added (bringing total H₂O₂ concentration to 29.3%); and bubble observations were started.

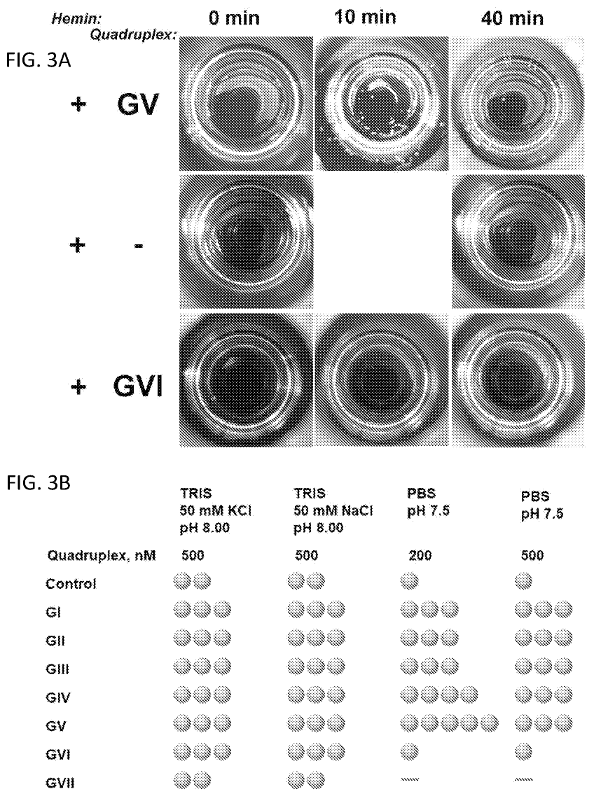

Addition of hydrogen peroxide is a “0 minutes” point as shown in FIGS. 3A, 5, and 12. After indicated time points, bubbles on the surface of the solution were observed either with a naked eye or a cell phone camera (e.g., an iphone XR). An equivalent volume of buffer instead of G-quadruplex solution was used in control samples.

### Catalase Activity Upon Target Detection

Split G-quadruplexes were prepared by denaturing of approximately 50 μM solutions of a left arm strand, a right arm strand, and a target in a PBS buffer (pH 7.5) at 95.0° C. for 5.0 minutes, slowly cooling down to room temperature, and equilibrating for additional 10 hours. Further sample preparation proceeded as described above for full G-quadruplexes GI-GVII but with split G-quadruplex/target hybrids. For controls, the samples with equivalent amounts of left and right arm strands but no target were equilibrated and treated in an identical fashion.

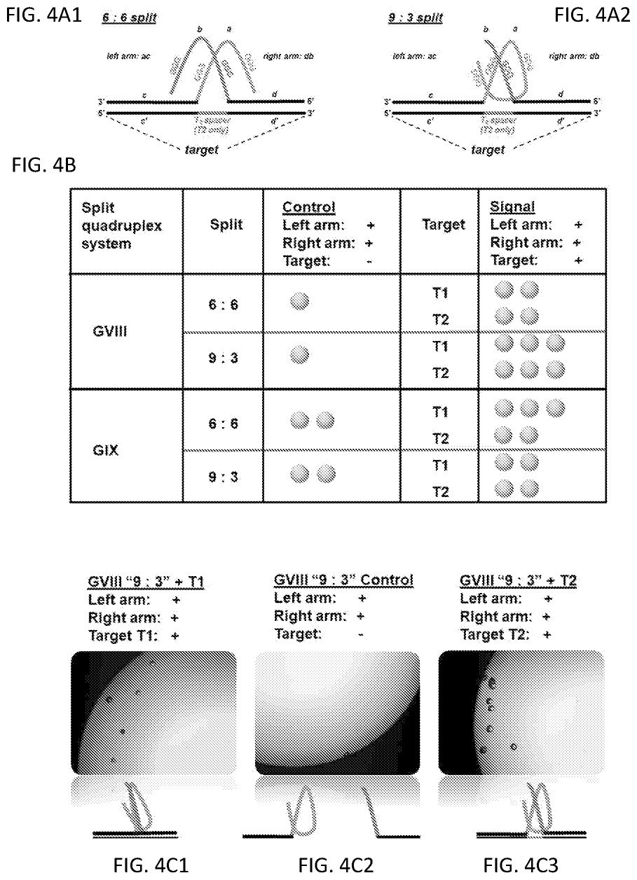

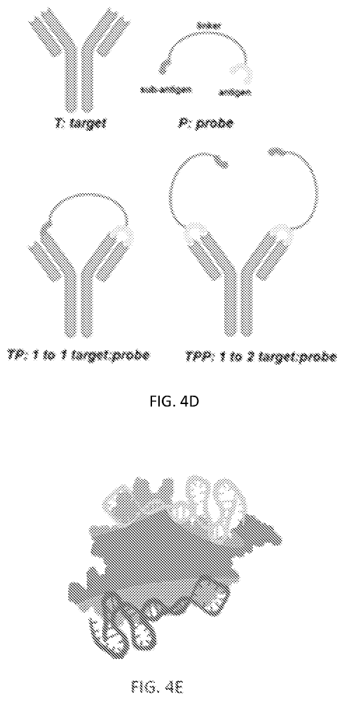

For microscopic observations (FIG. 4C), images were taken with BZ-X800 Analyzer (Keyance, Japan). The solutions were prepared in a 96-Well Plate and images were taken in Bright Field with a 4× objective. The reaction volumes were scaled down to yield the same concentrations as indicated above in a total volume of 300 μL. Sample preparation/observation timelines are equivalent to those used for naked eye observations.

### Quantitative Analysis

To enable quantitative analysis of various targets, the catalase activation system can be interfaced with stoichiometric mechanism based on negative cooperativity. According to this mechanism, a target concentration should be equivalent to probe concentration in order for two fragments to join together. The fragments are designed to form a catalase. Therefore, catalase will be activated only at a certain target concentration.

### Antibody Analysis

For antibody analysis (including quantitative analysis), peptide-based probes that will bind antibody can be designed (FIG. 4D). The linkers between two peptide fragments on each probe comprise oligonucleotide sequences so that when two probes are bound to antibody (in TPP), the catalase is activated (FIG. 4E). A linker refers to a part of a molecule that connects two functional parts.

### EXAMPLE 2

### G-Quadruplex Design and Environmental Conditions

The activatable catalases are composed of G-quadruplexes with at least three G-quartets because those form tight complexes with hemin. Thus, the oligonucleotide sequences with four consecutive stretches of 3-guanines are separated by 1-5 bases with a general formula NₐG_{b}NₐG_{b}NₐG_{b}NₐG_{b}Nₐ (SEQ ID NO: 35), wherein G=guanine; N=either guanine (G) or thymidine (T) or adenine (A) or cytosine (C); wherein each a in the sequence, independently, is a number between 0 and 5, and wherein each b in the sequence, independently, is either 3 or 4.

Some examples of sequences are included in Table 1. A core sequence (GI) is modified to generate three-G-quartet sequences GII-GV. As controls, two G-quadruplex sequences were designed that cannot form three-G-quartet structures because of insufficient number of guanines in continuous stretches (sequences GVI and GVII).

TABLE 1
Sequences of the oligonucleotides in Materials and Methods.

| | | |
|---|---|---|
| | | SEQ ID |
| Name | Sequence | NO: |

Full G-quadruplexes:

| | | |
|---|---|---|
| GI | 5′-GGG TTA GGG TTA GGG TTA GGG-3′ | 1 |
| GII | 5′-A GGG TTA GGG TTA GGG TTA GGG-3′ | 2 |
| GIII | 5′-ATT GGG TTA GGG TTA GGG TTA GGG-3′ | 3 |
| GIV | 5′-GGG TTA GGG TGT GGG TTA GGG-3′ | 4 |
| GV | 5′-GGG TTA GGG TCT GGG TTA GGG-3′ | 5 |
| GVI | 5′-A GG TTTT GG CA GGG TTTT GG T-3′ | 6 |
| GVII | 5′-A GGG TT GGG TGT GGG TT GG-3′ | 7 |

Split G-quadruplexes and target. Domains “a” are underlined; domains “b” are [symbol]
domains “c” are [symbol] , domains “d” are [symbol] , domains “c” [symbol] [symbol]
, domains “d” are

[symbol]

| | | |
|---|---|---|
| Target T1 | 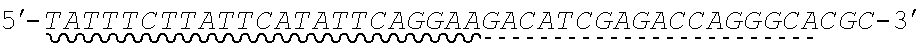 | 8 |
| Target T2 | 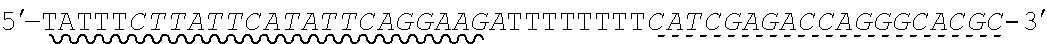 | 9 |
| GI 6:6 L | 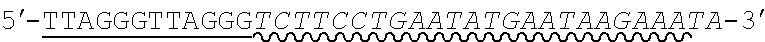 | 20 |
| GV 6:6 L | 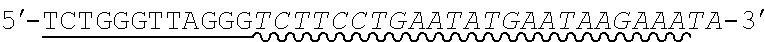 | 21 |
| GVIII 6:6 L | 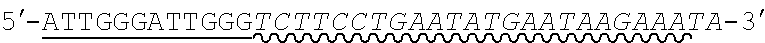 | 10 |
| GIX 6:6 L | 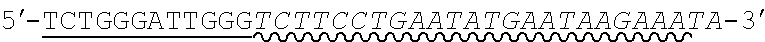 | 11 |
| GI 6:6 R | 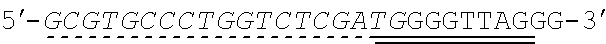 | 12 |
| GV | | |
| 6:6 R | | |
| GVIII | | |
| 6:6 R | | |
| GIX | | |
| 6:6 R | | |
| GI 9:3 L | 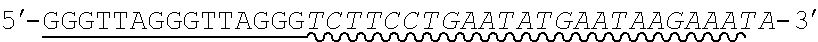 | 22 |
| GV 9:3 L | 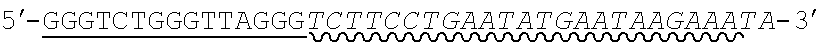 | 23 |
| GVIII 9:3 L | 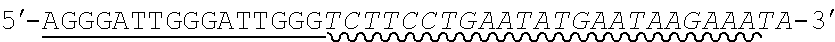 | 13 |
| GIX 9:3 L | 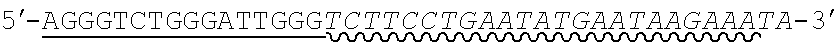 | 14 |
| GI 9:3 R | 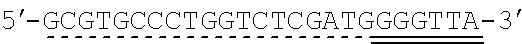 | 24 |
| GV | | |
| 9:3 R | | |
| GVIII 9:3 R | 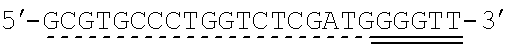 | 15 |
| GIX | | |
| 9:3 R | | |
| GVIII 9:3 split T control L GIX | 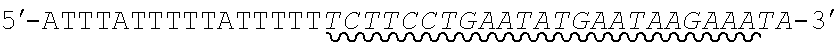 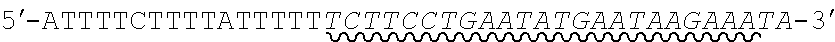 | 16 17 |
| 9:3 | | |
| split T | | |
| control L | | |
| 9:3 split T | 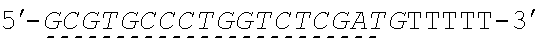 | 18 |
| control R | | |

Single stranded controls for polyacrylamide gel electrophoresis

| | | |
|---|---|---|
| 20-nt | 5′-GCCAGGAGAATGAGGTGGTC-3′ | 19 |
| PAGE | | |
| control | | |
| 21-nt | 5′-TAT TTC TTA TTC ATA TTC AGG-3′ | 25 |
| single | | |
| stranded | | |
| control | | |
| for PAGE | | |
| 22-nt | 5′-TAT TTC TTA TTC ATA TTC AGG A-3′ | 26 |
| single | | |
| stranded | | |
| control | | |
| for PAGE | | |
| 24-bp | 5′-TAT TTC TTA TTC ATA TTC AGG ATT-3′ | 27 |
| single | | |
| stranded | | |
| control | | |
| for PAGE | | |

Probes for quantitative analysis (FIG. S7) (target binding domains are
underlined, overlap regions are bold, G-quadruplex forming sequences are double-underlined)

| | | |
|---|---|---|
| Q1 | 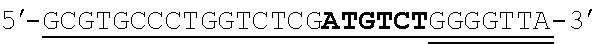 | 28 |
| Q2 | 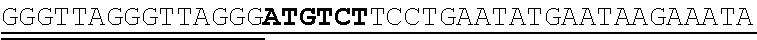 | 29 |

To summarize, sequences GI-GV are sequences with four consecutive stretches of 3-guanines, separated by between 1-5 bases of the general formula NₐG_{b}NₐG_{b}NₐG_{b}NₐG_{b}Nₐ (SEQ ID NO: 35), wherein G=guanine; N=either guanine (G) or thymidine (T) or adenine (A) or cytosine (C); wherein each a in the sequence, independently, is a number between 0 and 5, and wherein each b in the sequence, independently, is either 3 or 4. All sequences were derived from GI-GV by adding overhangs, shrinking or extending loops between guanine stretches, and/or changing nature and sequences of bases in loops, and can demonstrate catalase activity.

To select buffer conditions, it was noted that (i) the hemin-catalyzed hydrogen peroxide decomposition is the most efficient at pHs around 7 to 8, (ii) G-quadruplex folding requires a presence of Na⁺ or K⁺ ions, (iii) G-quadruplex/hemin DNAzymes show higher peroxidase activity at pH 7 to 8, (iv) optimal activity of catalases is observed over pH range of 7 to 11. Therefore, three buffering systems were evaluated: TRIS+50 mM KCl (pH 8.0), TRIS+50 mM NaCl (pH 8.0), and PBS (pH 7.5).

### EXAMPLE 3

### Catalase Activity of G-Quadruplexes

### Hemin Binding

To ensure that G-quadruplexes bind hemin (a condition for the activation of G-quadruplex DNAzymes), the Soret band on the hemin adsorption spectra was monitored. An increase in intensity and bathochromic shift was observed (FIG. 2) consistent with a hemin binding G-quadruplex scaffold. No significant changes to the Soret band were observed beyond the first 20 minutes of hemin/G-quadruplex interaction (for all quadruplexes except GVII). Therefore, at least 20 minutes of G-quadruplex·hemin equilibration time was allowed before introducing hydrogen peroxide into all the experimental conditions described herein.

### Catalase Activity

To evaluate the catalase activity of the quadruplexes, GI-GVII were exposed to hydrogen peroxide in the presence of hemin in the three buffering systems. To ensure hemin binding, G-quadruplex/hemin mixtures were equilibrated for 20 minutes before adding H₂O₂.

As shown in FIG. 3, it was observed that control experiments (i.e., those in the absence of any G-quadruplex) in both versions of TRIS buffer demonstrate substantially higher background bubbling levels compared to PBS. Furthermore, it was observed that quadruplexes GI-GV produce bubbles in amounts exceeding corresponding controls under all buffering conditions evaluated. Finally, it was shown that GVII (in all conditions) and GVI (in PBS) did not produce bubbles in amounts that observably exceed control levels.

### Correlation Between the Catalase Activity and G-Quadruplex Folding Topology.

Structure-function correlations for G-quadruplex hemin catalases need to take into account folding topology of G-quadruplexes. G-quadruplexes can fold into various topologies that are designated as parallel, antiparallel, or hybrid.

A G-quadruplex folding topology can be assessed via evaluation of circular dichroism (CD) properties of the folded quadruplexes. CD-spectra of quadruplexes GI-GVIII (FIG. 7) were evaluated and a conclusion was that the spectra of quadruplexes do enhance catalase activity of hemin (GI-GV) to conform to antiparallel folding topology.

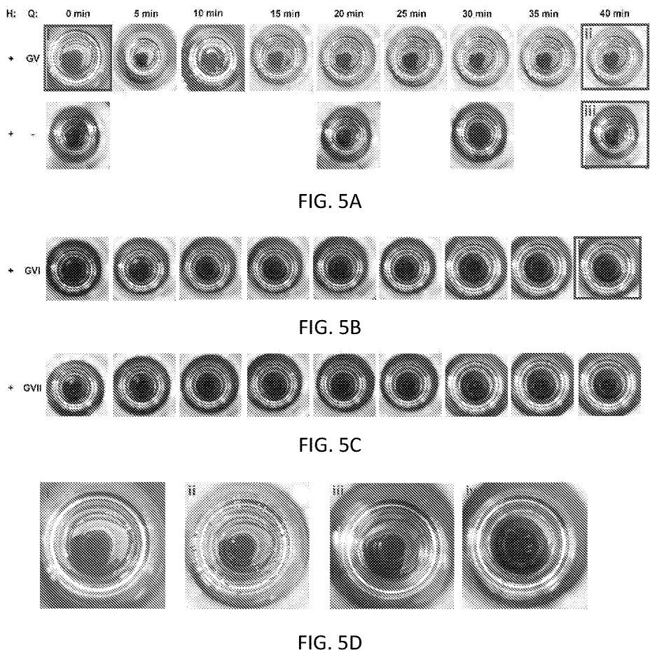

Evaluation of thermal folding profiles demonstrated mostly folded conformation of GI and GV over ambient temperature range (20-25° C.), a mixture of folded and unfolded confirmations for GII-GIV and GVI, and mostly unfolded state of GVII. On nondenaturing gel electropherograms (FIG. 6), bands for GI, GII, GIV, GV and GVII migrate father than corresponding non-structured strands of identical lengths. Migration of GIII and GVI does not differ from controls. Some variances observed between gels and thermal profiles (e.g. folded state for GVII is evident on gel but no folding appears on thermal profiles or no folding of GIII and GVI on gel but partial folding follows from the corresponding thermal profiles) may originate from low temperature of gel separations (4° C.) and/or the presence of glycerol in samples. Importantly, both experiments point towards consistently stable folded state of GV, a sequence that shows the highest catalase enhancement in PBS (see FIG. 5).

Rational development of G-quadruplex based catalases requires understanding of G-quadruplex structure-activity relationships. First, via non-denaturing PAGE, all the oligonucleotides GI, GII, GIV, GV, and GVII were demonstrated to fold into quadruplex structures (FIG. 6).

Next, CD spectra of all the G-quadruplexes demonstrated a connection between their folding topology and catalase activity (FIG. 7). Catalase-active quadruplexes GI-GV are noted to contain three common features: a maximum at 290-295 nm, a minimum at 262-267 nm (e.g., 265 nm), and a maximum at 240-250 nm. The features agree well with antiparallel folding topology.

Spectra of catalase inactive (in PBS) GVI and GVII do not contain all of the three features. Moreover, the CD spectra of GVI and GVII CD did not correlate with the three clearly defined topological systems (parallel, antiparallel, or hybrid/3+1), indicating either to a mixture of conformations or to partially folded states. Kinetically, quadruplexes GI-GV appear to bind hemin almost instantly while GVI and GVII were slower to bind (FIG. 2).

Without being bound by any theory, the differences in catalytic behavior of GI-GV and GVI-GVII could be a result of two sources: differences in hemin binding kinetics and variations in folding topology.

### EXAMPLE 4

### Activatable G-Quadruplex Catalases

To enable a target-triggered bubble production, probes were engineered that form a full quadruplex only in the presence of a target. The probes included two pieces of quadruplex and form a full quadruplex that is necessary for catalase activity. The probes that are not bound to a target are unlikely to form a quadruplex.

Upon bringing the fragments into proximity, the fragments assembled into a quadruplex structure. To make the quadruplex assembly contingent on the presence of a target oligonucleotide strand, G-quadruplexes were split into left (L) and right (R) “arms” (FIG. 4A, domains a and b) and the “arms” were extended, with target recognition domains (c and d) that are complementary to fragments c′ and d′ on a target. Therefore, in the presence of target, c/c′ and d/d′ hybridization brings domains a and b in a close proximity, triggering a G-quadruplex assembly.

To refine the design, different split types were evaluated, in particular a symmetric split (6:6) and an asymmetrical split (9:3). In the symmetric split (6:6), both arms contained two three-guanine stretches. In contrast, for the asymmetrical split, the right arm contained one three-guanine stretch and the left arm contained three three-guanine stretches (FIG. 4A). To follow the design considerations, two split G-quadruplex systems (GVIII and GIX) were obtained (see Table 1 and FIG. 4A) and two target conformations were constructed (i.e., in target T1, domains c′ and d′ are adjacent to each other while in Target T2, domains c′ and d′ are separated by an 8-nucleotide “spacer”).

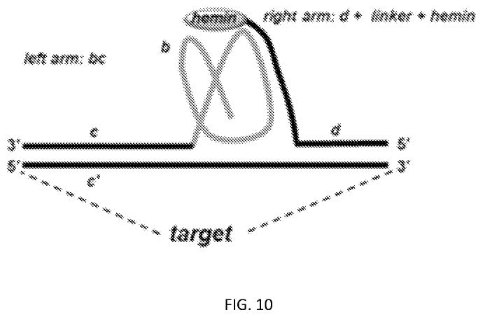

Evaluation of different activatable catalase configurations (FIG. 4) demonstrated that the highest signal-to-background ratio in response to target binding was observed for an asymmetrical GVIII system. Asymmetrically split G-quadruplexes demonstrated higher peroxidase activity and formed tighter complexes with protoporphyrin IX (e.g., a compound structurally similar to hemin. FIG. 7 shows a preliminary insight into folding topology of a GVIII 9:3 split and indicates an antiparallel arrangement that is in agreement with findings of catalase activity of antiparallel full quadruplexes GI-GV. To enable precise positioning on hemin next to quadruplex, an intact quadruplex modified with domain C and synthesized hemin-modified Domain D was obtained through EDC/NHS coupling (FIG. 10).

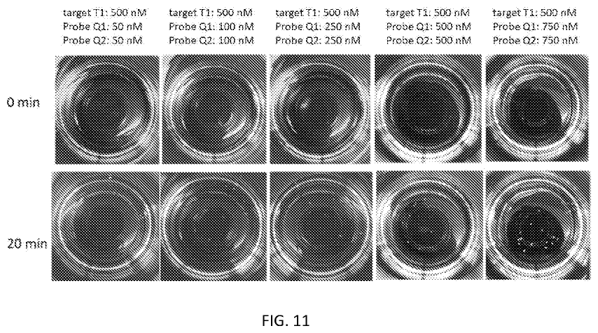

Furthermore, the catalase system was expanded into a field of quantitative analysis. The target-assembly activatable system (split) is compatible with a stoichiometric approach for quantitative analysis of biomolecules (see, e.g., Adegbenro, A.; Coleman, S.; Nesterova, I. V. (2022) Stoichiometric Approach to Quantitative Analysis of Biomolecules: the Case of Nucleic Acids, Anal. Bioanal. Chem., 414, 1587-1594 and Debnath, M.; Farace, J. M.; Johnson, K. D.; Nesterova, I. V. (2018) Quantitation without Calibration: Response Profile as an Indicator of Target Amount, Anal. Chem., 90, 7800-7803, herein incorporated by reference in their entireties. The approach enables naked eye quantitative analysis of a target T1 at 500 nM level (FIG. 11).

In conclusion, G-quadruplex·hemin constructs are capable of mimicking protein catalases and, as such, activate hydrogen peroxide decomposition. The reaction produces molecular oxygen that, when released in the form of bubbles, serves as a visual signal readout platform for molecular recognition events.

Overall, the significance of activatable catalases can extend beyond pure analytical applications into therapeutic areas where reactive oxygen species scavengers are in a high demand.

### EXAMPLE 5

### Development of an Artificial Catalase Based on a G-Quadruplex Hemin Assembly

Artificial catalases can be formulated based on G-quadruplex·hemin constructs. Nucleic acids containing a stretch of guanines can fold into quadruplex structures (G-quadruplexes) composed of stacked square planar four-guanines structures called G-quartets (see, e.g., Mergny, J.-L.; Sen, D. DNA Quadruple Helices in Nanotechnology. Chem. Rev. 2019, 119, 6290-6325, incorporated herein by reference). Depending on nucleic acid sequences and environments (e.g., buffer, pH, ionic strength), G-quadruplexes can fold into different topologies. Both quadruplex folding state and folding topology can affect catalytic activity of G-quadruplex·hemin constructs (as demonstrated for peroxidase mimics).

A requirement for a “yes/no” readout format is zero signal in the absence of an appropriate trigger (e.g., a defined target amount for quantitative analysis). Therefore, conditions that can provide a zero background in the absence of assembled G-quadruplex but will enable a maximized response when G-quadruplex is assembled are important.

### Buffer Selection

Selection of buffer conditions can take into account (i) the hemin-catalyzed hydrogen peroxide decomposition is the most efficient at pHs around 7-8; (ii) G-quadruplex folding in the presence of Na+ or K+ ions for stabilizing; (iii) G-quadruplex/hemin DNAzymes demonstrating higher peroxidase activity at pH 7-8; and (iv) optimal activity of catalases being over a pH range of 7-11. Therefore, an appropriate buffering system can be designed provide capacity around pH of 7 to 8 and contain Na+ and/or K+.

Evaluation of TRIS supplemented with NaCl and with KCl against PBS buffer demonstrated that PBS at pH 7.5 provided the lowest background (e.g., minimized bubble formation in the absence of quadruplex). Other buffers can provide true “zero background bubble formation” as a selection criterion, such as buffers with a pH 7-8 buffering range (e.g. MOPS, BES, HEPES) supplemented with 50-100 mM Na+ and or K+ at pH 7-8. Hydrogen peroxide can be exposed in the presence of hemin to various conditions and the appearance of bubbles can be monitored.

### Core Quadruplexes Selection and Evaluation

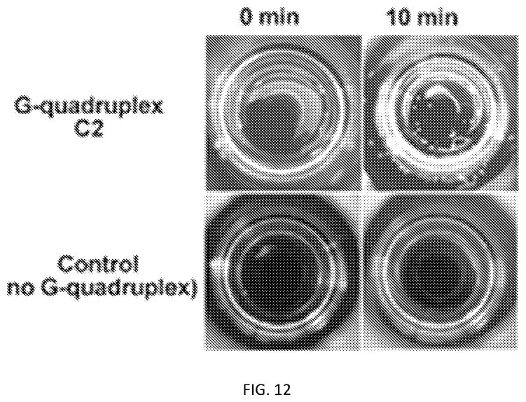

Thereafter, core quadruplex evaluations can proceed by utilizing appropriate buffering conditions, for instance using three core oligonucleotide sequences (C1-C3) established catalase mimics (see FIG. 12).

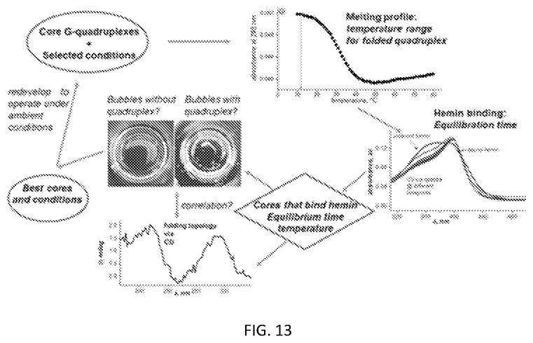

To establish folding state/catalase activity correlation and folding topology/catalase activity correlation, each core oligonucleotide sequence can be evaluated in buffering conditions as described herein via the following analyses and as illustrated in FIG. 13.

First, evaluations can analyze the folding state via UV-vis melting studies and a temperature range where quadruplex is folded can be established. Second, using optimal temperatures, hemin binding dynamics can be assessed via UV-vis to identify quadruplexes that bind hemin. This can also establish binding time for hemin binders.

Third, for selected temperature and hemin binding time, bubble production in the presence H₂O₂ can be evaluated. For instance, controls including all of the components except G-quadruplex can be evaluated side by side. As a starting point, 200 nM G-quadruplex concentrations can be used and cores with ultimate catalase activities can be selected.

Folding topology via CD can be evaluated to understand the folding topology/activity correlation, thus establishing rational design of effective G-quadruplex·hemin catalases. For active cores that operate at low (below ambient) temperatures, quadruplexes can be constructed to ensure higher stability, and therefore to make them folded at higher temperatures (e.g., a conventional room temperature).

Various combinations of buffers/cores providing an acceptable signal to background ratios at room temperature can be identified. Further, a correlation between catalase activity and quadruplex folding state and topology can be established. Optimization of the experimental parameters such as G-quadruplex/hemin ratios and H₂O₂ concentrations. Finally, the lowest quadruplex concentrations that produce observable difference in bubble production compared to controls can be established, for instance detection of low nanomolar concentrations of quadruplexes at 200 nM levels.

### EXAMPLE 6

### Structural Modifications of G-Quadruplex·hemin Catalases

Generally, catalytic rates of DNAzymes are lower than of protein analogs. Without being bound by any theory, it is believed that bubble formation requires higher rates of hydrogen peroxide decomposition to boost up gas accumulation. To maximize the rate, a core quadruplex can be modified with fragments that can enhance catalase activity. For instance, histidine (i.e., His56 of native H. pylori catalase or His64 of P. Vitale catalase) is critical for catalytic activity as a promoter of prototropic isomerization of H₂O₂ and when the residue is mutated, oxidation rates in protein catalases are lower.

Accordingly, including a proton acceptor near hemin binding site can likely increase the catalytic activity of hemin. Further, including nucleobases (proton acceptors) in proximity to the binding site of hemin can enhance peroxidase activity of G-quadruplex/hemin constructs and this concept can be applied to develop DNAzyme catalases. Specifically, two proton acceptor groups can be evaluated: nucleobases and imidazole (a side chain in histidine).

### Catalytic Activity Enhancement by Nucleobases

Recent reports indicate that dA's or dA-runs enable enhanced peroxidase activity of G-quadruples/hemin DNAzymes. Therefore, for a given core structure, a series of variants can be designed with dA and short dA-runs positioned in 3′ flanking region (known to bind hemin) or in adjacent loops (see Table 2). Catalase activity of constructs can be monitored and signal-to-background ratio in terms of bubble production can be evaluated.

TABLE 2
Modifications with dA for core quadruplex C1
(modifications in bold)

| Core | Sequence 5′→3′ | SEQ ID NO: |
|---|---|---|
| C1 | GGGTAGGGCGGGTTGGG (unmodified) | 30 |
| | GGGTAGGGCGGGTTGGG A | 31 |
| | GGGTAGGGCGGGTTGGG AAA | 32 |
| | GGGTAGGGCGGGTTAGGG | 33 |
| | GGGTAGGGCGGGAAGGG | 34 |

TABLE 3
Modifications with dA for core quadruplex C2
(modifications in bold)

| Core | Sequence 5′→3′ | SEQ ID NO: |
|---|---|---|
| C2 | AGGGTTAGGGTTAGGGTTAGGG | 36 |
| | (unmodified) | |
| | AGGGTTAGGGTTAGGGTTAGGG A | 38 |
| | AGGGTTAGGGTTAGGGTTAGGG AAA | 39 |
| | AGGGTTAGGGTTAGGGTTAAGGG | 40 |

TABLE 4
Modifications with dA for core quadruplex C3
(modifications in bold)

| Core | Sequence 5′→3′ | SEQ ID NO: |
|---|---|---|
| C3 | TGGGTTGGGTTGGGTTGGGT | 37 |
| | (unmodified) | |
| | TGGGTTGGGTTGGGTTGGGT A | 41 |
| | TGGGTTGGGTTGGGTTGGGT AAA | 42 |
| | TGGGTTGGGTTGGGTTAGGGT | 43 |
| | TGGGTTGGGTTGGGTTAAGGGT | 44 |

### Imidazole Modifications

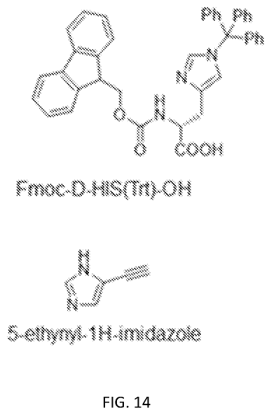

Without being bound by any theory, it is believed that imidazole (histidine's residue) is critical for catalytic activity of protein catalases. Natural nucleic acids do not contain imidazole fragments. To enable catalytic enhancement in our mimics, G-quadruplexes can be modified at 3′ flanking region or in adjacent loops with imidazole fragment. To synthesize the modified version, an amino-protected histidine (e.g., Fmoc-D-His (Trt)-OH, see FIG. 14) can be reacted with amino-modified oligonucleotide through EDC/NHS coupling. The imidazole-modified oligonucleotide can be isolated via HPLC, characterized with MS, and evaluated with respect to bubble production against appropriate controls and an unmodified core version.

### EXAMPLE 7

Evaluation of System Compatibility with Relevant Matrices

The instant example evaluates operation of the catalase system under relevant environments. Overall, nucleic acid-based sensors are capable for use in clinically-relevant samples. Two relevant systems can be evaluated: i) blood serum (e.g., a matrix for antibody markers) and ii) simulated nasal mucus (e.g., a matrix for oligonucleotide markers).

First, for both environments, a dilution that enables zero background level can be established. For blood serum evaluation, buffer/serum mixtures with 90/10 to 10/90 ratios can be prepared. Similarly, the dilution levels for nasal mucus can be evaluated. For optimized background conditions, the performance of best quadruplex systems can be evaluated.

The relevant concentration of antibodies in blood sample can provide a micromolar range. Since the described catalase systems can function at a 200 nM range, the system can provide antibody analysis.

### EXAMPLE 8

### Engineering an Equipment-Free Stoichiometric Approach for Quantitative Analysis of Oligonucleotides and Antibody Targets

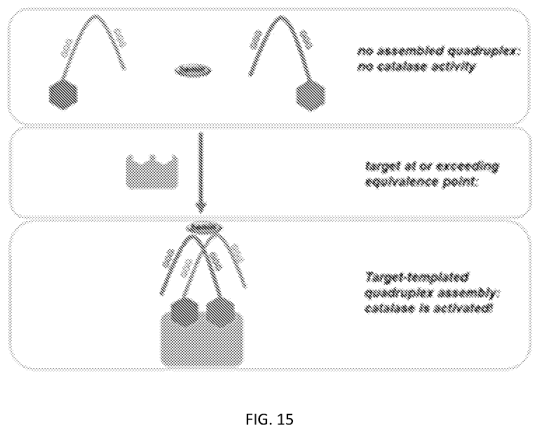

The instant example provide analysis of the mechanism for transducing stoichiometric equivalence point into catalase activation. In particular, the catalase can be split with its assembly made into an active state contingent on a target recognition. The G-quadruplex/hemin system can be split into two fragments wherein the separated fragments may be unlikely to form an active enzyme. To assemble into an active enzyme, the fragments can then be placed in a close proximity (FIG. 15).

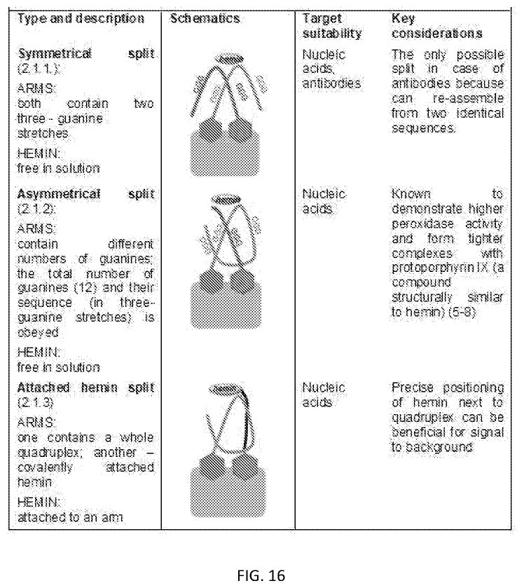

For instance, in G-quadruplex hemin peroxidases, split type impacts the effectiveness G-quadruplex reassembly. Accordingly, a split can be designed according to the instant example that i) does not give background in the absence of target and ii) turns on triggered by target at defined concentration. Quadruplex split strategies are found in FIG. 16.

A stoichiometric model can be implemented for oligonucleotide targets to make quadruplex assembly contingent on reaching the equivalence point. Specifically, a G-quadruplex sequence can be split into left (L) and right R “arms” (FIG. 4A, domains a and b) and extend the “arms” with target recognition domains (FIG. 4A, domains c and d). Domains c and d are designed to be complementary to fragments c′ and d′ on a target. Therefore, in presence of target, c/c′ and d/d′ hybridization brings domains a and b in a close proximity triggering a G-quadruplex assembly. The strategy can be applied towards symmetrical, asymmetrical, and attached hemin splits.

In order to unable the stoichiometric capability, domains c′ and d′ can overlap. In this case, if both probes present equimolarly and one of the probes binds to target the affinity for the second binding event is lower than that for the first one (“occupied” overlapping region will destabilize second probe/target duplex). Therefore, the second probe will not bind to the same target and, as a result, the G-quadruplex will not reassemble, until each probe concentration exceeds the target level. In this case, the hybrid of target with two probes can form triggering a G-quadruplex assembly. This strategy can be used for quantitative analysis (FIG. 11). The capabilities demonstrated in FIG. 16 can provide i) systems that give zero background when target concentration is below pre-defined stoichiometric value; ii) systems that generate visible bubbling when target reaches a pre-defined stoichiometric value, and iii) limits of detection with respect to the lowest target concentration.

### Symmetric Splits

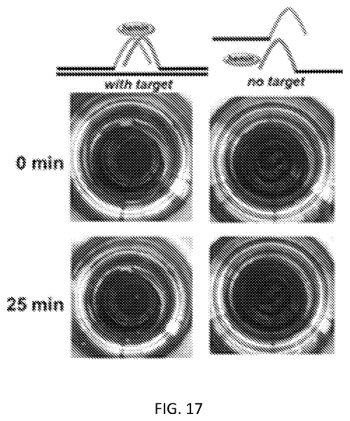

Efficiently functioning symmetrical splits can be developed as a mechanism for generating signal in antibodies. As shown in FIG. 17, symmetric splits can provide efficacy. Further efficacy can be improved via quantitation levels that are decreased below demonstrated 500 nM and also via an increase in bubble production.

### Achieving Ultimate Sensitivity of Activatable Catalases

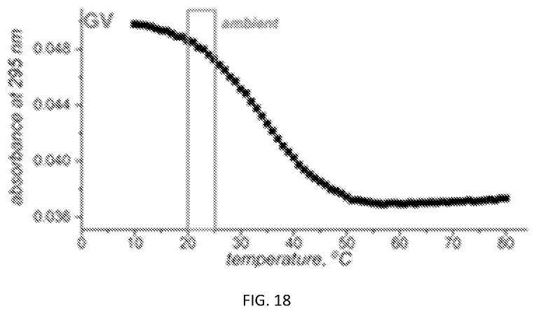

Various strategies can also be employed to maximize sensitivity when splitting quadruplexes. For instance, decreasing target concentration can ensure thermodynamic stability of assembled split structure. Thus, as shown in FIG. 17, the “parent” (not split) quadruplex melting profile indicates that it is assembled but its melting temperature is just above ambient (see FIG. 18). Although a melting profile of the split system was not established, it is believed that its thermodynamic stability is lower than the whole unsplit “parent” quadruplex. It appears that that only a fraction of split quadruplex was assembled FIG. 17, suggesting that ensuring thermodynamic stability of split quadruplex is the key for improving sensitivity of the system.

Thermodynamic stability of G-quadruplexes can be modulated via structure and length of loops and overhangs and through adding additional guanines/G-quartets and/or via minor groove tetrad-induced stabilization. To demonstrate the stability, melting profiles of modified splits through CD and UV-vis can be evaluated. Structures that are folded at room temperature can be further developed.

### Decreasing Background Bubbling in Split Quadruplex Systems

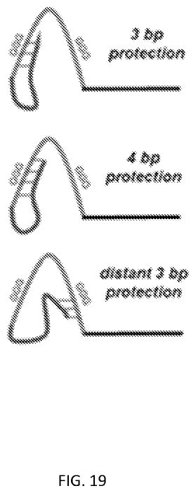

Without being bound by any theory, background bubbling can be observed as an off-target assembly of quadruplexes (G-stretches of different arms interact without target present). To eliminate the non-target assembly, quadruplexes can be modified with protective hairpins (FIG. 19). Hairpins can be designed that are stable enough to prevent assembly of quadruplexes without template but do not interfere with target-templated quadruplex assembly. A target-templated assembly refers to a mechanism by which target recognition results in assembly of an active catalase. To accomplish the task, a series of hairpins with different stem length/GC content can be evaluated to evaluate those that satisfy our yes/no binary response criteria. An example set of hairpins is shown in FIG. 19.

A system in which one of the probes “protected” with a hairpin and another unprotected can be evaluated. While such system will not be absolutely symmetric as required for antibody detection, it can provide detection modality for targets that do allow non-identical probes.

### Asymmetrical Splits

In general, asymmetrically split G-quadruplexes can demonstrate higher peroxidase activity and form tighter complexes with protoporphyrin IX (a compound structurally similar to hemin) upon assembly. Various asymmetrical splits types can be evaluated as quadruplex split type affect efficiency of quadruplex assembly. The asymmetrical splits types refer to the number of guanines on separated fragments (e.g., 9:3; 8:4, 5:3, etc.). As shown in FIG. 4C, activation of catalase activity in a 9:3 split quadruplex C2 in the presence of target was observed and proposed systematic evaluation of splits will yield more efficient split systems.

For both symmetric splits and asymmetrical splits, sensitivity of catalase upon reassembly can be evaluated as well as development of lowest background by decreasing off-target bubbling. For the asymmetrical split, quadruplexes can be modified to ensure that folded state upon reassembly (at ambient conditions). For decreasing background bubbling, the quadruplexes can be protected with hairpins. In case of asymmetric split, the protection of one probe may be beneficial for achieving zero background/high catalase activity (yes/no) switch.

### Attached Hemin Split

Without being bound by any theory, precise positioning on hemin next to quadruplexes can contribute towards more efficient catalase activation. An intact quadruplex modified with arm c can be obtained and hemin-modified arm d can be synthesized (FIG. 10). To synthesize hemin-modified arm d, an amino-modified arm d can be obtained and modified with hemin through EDC/NHS coupling using one of hemin's carboxylic groups. The conjugate can be isolated via HPLC, and characterized with LC/MS.

### EXAMPLE 9

### Design and Characterization of Models for Quantitative Analysis of Antibody Targets

A stoichiometric quantitative analysis platform can be used for quantitation of IgG antibodies. To enable equivalence point detection, a negative cooperativity system similar to one discussed for oligonucleotide targets was employed. As recognition fragments, peptides that specifically bind the antibodies were employed. The operational principles of stoichiometric system are illustrated and discussed in FIG. 4D. The system successfully operates in negative cooperativity mode and enables stoichiometric response profiles.

For antigens, the linker between antigen and sub-antigen domains can be oligonucleotide duplexes. This provides rigidity needed for competitive binding in the stoichiometric scheme and also allows incorporating functional assemblies (e.g., DNAzyme).

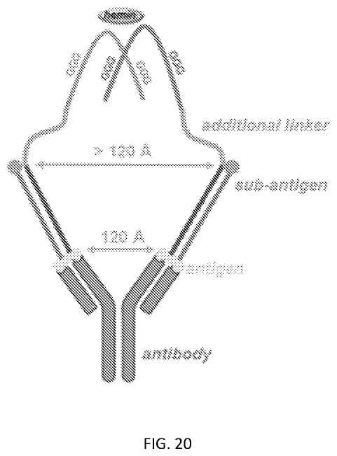

An Anti-HIV-1 p17 murine monoclonal IgG1 b12 antibody targeted against the p17 proteins HIV1 can be utilized for probe development. An activation scheme can be provided to signal quantitative threshold for the antibody. Since the antibody binding scheme is based on two identical probes, the signaling that is activated upon target binding both probes can be based on symmetric splits as described herein (see, e.g., FIG. 20).

Unlike oligonucleotide targets, antibodies' binding sites are generally separated. Therefore, to enable efficient quadruplex reassembly, two split parts need to come into a contact. Thus, linkers can be optimized between the major dsDNA linker that separates peptide domains and G-quadruplex parts (e.g., the “additional linker” in FIG. 20) regarding flexibility and length. Regarding flexibility, flexible linkers can be evaluated based on polyethylene glycol (PEG) chains and rigid linkers can be evaluated based on nucleic acids. The criteria for linker performance can be an efficient catalase activation upon achieving stoichiometric ratio between target and probes.

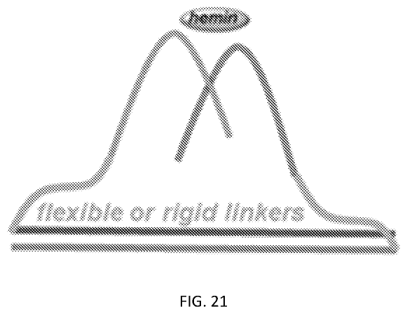

To streamline the linker development and make it more efficient, “distant” models can be developed using a rigid oligonucleotide double helix to imitate distance. The antibodies have two identical binding sites separated by about 12-17 nm. Therefore, the split G-quadruplex fragments can be separated by a distance greater than 120 Å. A ds DNA of 35 bp has a length of 119 A (according to the X-ray structure (PDB: 1HZH)). A double stranded DNA of 50 bp that has two overhangs on opposite sides can be designed since the distance between fragments will actually be longer (due additional length from probe linkers as shown in FIG. 20. The overhangs can comprise split quadruplex fragments separated from the duplex portion through a linker (see FIG. 21).

As quadruplex fragments, the symmetrically split systems described can be evaluated for performance of different hybrids in the presence of free hemin and H₂O₂ with a criteria of maximized bubble amount. Thereafter, the split quadruplex/linker system can be transferred to peptide-based probes suitable for oligonucleotide binding.

1. A method to detect a target in a sample, the method comprising:
a) obtaining a DNAzyme that catalyzes hydrogen peroxidase decomposition;
b) contacting the DNAzyme to the sample to produce a reaction, wherein bubbles are produced via the reaction when the target is present in the sample.

2. The method of claim 1, wherein the target is selected from a group consisting of a biomarker, an oligonucleotide, an antibody, a peptide and a small molecule.

3.-7. (canceled)

8. The method of claim 1, wherein the sample is a biological sample.

9.-12. (canceled)

13. The method of claim 1, wherein the DNAzyme is a G-quadruplex·hemin.

14. The method of claim 13, wherein the G-quadruplex comprises an oligonucleotide.

15. The method of claim 14, wherein the oligonucleotide comprises a sequence comprising NaGbNaGbNaGbNaGbNa (SEQ ID NO: 35),
wherein G represents guanine,
wherein N represents one of guanine (G), thymidine (T), adenine (A), cytosine (C),
wherein each a in the sequence, independently, is a number between 0 and 5, and
wherein each b in the sequence, independently, is either 3 or 4.

16. The method of claim 1, wherein the method is performed at a pH between 7 and 11.

17.-20. (canceled)

21. A kit for detection of a target in a sample, the kit comprising
a swab for the sample,
a DNAzyme that catalyzes hydrogen peroxidase decomposition, and
a container for reacting the sample with the DNAzyme, wherein the sample and the DNAzyme are capable of being contacted in the container to form bubbles.

22. The kit of claim 21, wherein the target is selected from a group consisting of a biomarker, an oligonucleotide, an antibody, a peptide and a small molecule.

23. The kit of claim 21, wherein the target is a biomarker.

24. The kit of claim 21, wherein the target is an oligonucleotide.

25. The kit of claim 21, wherein the target is an antibody.

26. The kit of claim 21, wherein the target is a peptide.

27. The kit of claim 21, wherein the target is a small molecule.

28. The kit of claim 21, wherein the sample is a biological sample.

29.-32. (canceled)

33. The kit of claim 21, wherein the DNAzyme is a G-quadruplex·hemin.

34. The kit of claim 33, wherein the G-quadruplex comprises an oligonucleotide.

35. The kit of claim 34, wherein the oligonucleotide comprises a sequence comprising NaGbNaGbNaGbNaGbNa (SEQ ID NO: 35), wherein G represents guanine,
wherein N represents one of guanine (G), thymidine (T), adenine (A), cytosine (C),
wherein each a in the sequence, independently, is a number between 0 and 5, and
wherein each b in the sequence, independently, is either 3 or 4.

36. The kit of claim 34, wherein the oligonucleotide comprises a confirmation in a folded state.

37. The kit of claim 34, wherein the oligonucleotide comprises a confirmation in an unfolded state.
